(19) United States
(12) Patent Application Publication
(10) Pub. No.: US 2006/0019975 A1
(43) Pub. Date: Jan. 26, 2006

(54) Novel piperidyl derivatives of quinazoline and isoquinoline

(75) Inventors: John Michael Humphrey, Mystic, CT (US); Thomas Allen Chappie, Old Lyme, CT (US); Spiros Liras, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/178,104

(22) Filed: Jul. 8, 2005

Related U.S. Application Data
(60) Provisional application No. 60/590,943, filed on Jul. 23, 2004.

(51) Int. Cl. A61K 31/519; A61K 31/4709; C07D 403/04; C07D 401/04

(52) U.S. Cl. 514/266.22; 514/310; 544/284; 546/148

(57) ABSTRACT

The invention pertains to new piperidyl-substituted quinazoline and isoquinoline derivatives that serve as effective phosphodiesterase (PDE) inhibitors. The invention also relates to compounds that are selective inhibitors of PDE10. The invention further relates to intermediates for preparation of such compounds; pharmaceutical compositions comprising such compounds; and the use of such compounds in methods for treating certain central nervous system (CNS) or other disorders.

This application claims priority under 35 U.S.C 119 of U.S. Provisional 60/590,943 filed Jul. 23, 2004. The entire contents of the prior application are incorporated herein by reference.

### FIELD OF THE INVENTION

The invention pertains to new piperidyl-substituted quinazoline and isoquinoline derivatives that serve as effective phosphodiesterase (PDE) inhibitors. The invention also relates to compounds that are selective inhibitors of PDE10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### BACKGROUND OF INVENTION

Phosphodiesterases (PDEs) are a class of intracellular enzymes involved in the hydrolysis of the nucleotides cyclic adenosine monophosphate (cAMP) and cyclic guanosine monophosphates (cGMP) into their respective nucleotide monophosphates. The cyclic nucleotides cAMP and cGMP are synthesized by adenylyl and guanylyl cyclases, respectively, and function as intracellular second messengers regulating a vast array of intracellular processes particularly in neurons of the central nervous system. In neurons, this includes the activation of cAMP and cGMP dependent kinases and subsequent phosphorylation of proteins involved in acute regulation of synaptic transmission as well as in neuronal differentiation and survival. The complexity of cyclic nucleotide signaling is indicated by the molecular diversity of the enzymes involved in the synthesis and degradation of cAMP and cGMP. There are ten families of adenylyl cyclases, two of guanylyl cyclases, and eleven of phosphodiesterases. Furthermore, different types of neurons are known to express multiple isozymes of each of these classes, and there is good evidence for compartmentalization and specificity of function for different isozymes within a given cell.

A principal mechanism for regulating cyclic nucleotide signaling is by phosphodiesterase-catalyzed cyclic nucleotide catabolism. There are eleven known families of PDEs encoded by 21 different genes. Each gene typically yields multiple splice variants that further contribute to the isozyme diversity. The PDE families are distinguished functionally based on cyclic nucleotide substrate specificity, mechanism(s) of regulation, and sensitivity to inhibitors. Furthermore, PDEs are differentially expressed throughout the organism, including in the central nervous system. As a result of these distinct enzymatic activities and localization, different PDE isozymes can serve distinct physiological functions. Furthermore, compounds that can selectively inhibit distinct PDE families or isozymes may offer particular therapeutic effects, fewer side effects, or both.

PDE10 is identified as a unique family based on primary amino acid sequence and distinct enzymatic activity. Homology screening of EST databases revealed mouse PDE10A as the first member of the PDE10 family of PDEs (Fujishige et al., J. Biol. Chem. 274:18438-18445, 1999; Loughney, K. et al., Gene 234:109-117, 1999). The murine homologue has also been cloned (Soderling, S. et al., Proc. Natl. Acad. Sci. USA 96:7071-7076, 1999) and N-terminal splice variants of both the rat and human genes have been identified (Kotera, J. et al., Biochem. Biophys. Res. Comm. 261:551-557, 1999; Fujishige, K. et al., Eur. J. Biochem. 266:1118-1127, 1999). There is a high degree of homology across species. The mouse PDE10A1 is a 779 amino acid protein that hydrolyzes both cAMP and cGMP to AMP and GMP, respectively. The affinity of PDE10 for cAMP (Km=0.05 μM) is higher than for cGMP (Km=3 μM). However, the approximately 5-fold greater Vmax for cGMP over cAMP has lead to the suggestion that PDE10 is a unique cAMP-inhibited cGMPase (Fujishige et al., J. Biol. Chem. 274:18438-18445, 1999).

The PDE10 family of polypeptides shows a lower degree of sequence homology as compared to previously identified PDE families and has been shown to be insensitive to certain inhibitors that are known to be specific for other PDE families. U.S. Pat. No. 6,350,603.

PDE10 also is uniquely localized in mammals relative to other PDE families. mRNA for PDE10 is highly expressed only in testis and brain (Fujishige, K. et al., Eur. J. Biochem. 266:1118-1127, 1999; Soderling, S. et al., Proc. Natl. Acad. Sci. 96:7071-7076, 1999; Loughney, K. et al., Gene 234:109-117, 1999). These initial studies indicated that within the brain PDE10 expression is highest in the striatum (caudate and putamen), n. accumbens, and olfactory tubercle. More recently, a detailed analysis has been made of the expression pattern in rodent brain of PDE10 mRNA (Seeger, T. F. et al., Abst. Soc. Neurosci. 26:345.10, 2000) and PDE10 protein (Menniti, F. S., Stick, C. A., Seeger, T. F., and Ryan, A. M., Immunohistochemical localization of PDE10 in the rat brain. William Harvey Research Conference ‘Phosphodiesterase in Health and Disease’, Porto, Portugal, Dec. 5-7, 2001).

A variety of therapeutic uses for PDE inhibitors have been reported including obtrusive lung disease, allergies, hypertension, angina, congestive heart failure, depression and erectile dysfunction (WO 01/41807 A2).

The use of selected benzimidazole and related heterocyclic compounds in the treatment of ischemic heart conditions has been disclosed based upon inhibition of PDE associated cGMP activity. U.S. Pat. No. 5,693,652.

U.S. Patent Application Publication No. 2003/0032579 discloses a method for treating certain neurologic and psychiatric disorders with the selective PDE10 inhibitor papaverine. In particular, the method relates to psychotic disorders such as schizophrenia, delusional disorders and drug-induced psychosis; to anxiety disorders such as panic and obsessive-compulsive disorder; and to movement disorders including Parkinson's disease and Huntington's disease.

Thus, in their role as second messengers in intracellular signaling events, cAMP and cGMP affect a wide array of processes including neurotransmission and enzyme activation. Intracellular levels of these chemicals are largely maintained by two classes of enzymes in response to other cellular stimuli. The adenylyl and guanylyl cyclases catalyze the formation of cAMP and cGMP thereby raising their concentrations and activating certain signaling events. The phosphodiesterases (PDE's) catalyze the degradation of cAMP and cGMP which results in termination of the signal.

Signal enhancement via elevation of cyclic nucleotide concentration can be induced through employment of PDE inhibitors. Opportunities exist for the use of such PDE inhibitors as therapies for the prevention or treatment of diseases linked to abnormal cell signaling processes.

### SUMMARY OF THE INVENTION

This invention relates to a compound having the formula 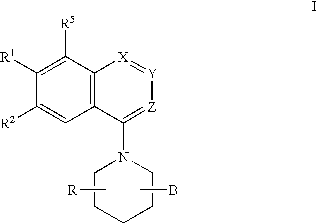
or a pharmaceutically acceptable salt, solvate or prodrug thereof,
wherein X, Y and Z are each independently N or CH, provided that at least one of X, Y and Z must be N or CH and provided that when Z is nitrogen, Y is CH; and when Y is nitrogen, X is nitrogen and Z is CH;
wherein R¹, R² and R⁵ are independently H, halogen, CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹, R² and R⁵ are independently alkoxy, alkenyloxy or alkyl, R¹ and R² or R¹ and R⁵ may optionally be connected to form a 5 to 8 membered ring; and when R¹, R² and R⁵ are —NR³R⁴, R³ and R⁴ may optionally combine with the nitrogen in which they are attached to form a 5 to 8 membered ring;
wherein R is H, —COOR³, —CONR³R⁴, —COR⁴, —NR³R⁴, —NHCOR³, —OH, —HNCOOR³, —CN, —HNCONHR⁴ (C₁-C₆)alkyl or (C₂-C₆) alkoxy;
wherein R³ and R⁴ are independently H, (C₁-C₆) alkyl, alkenyl, aryl or substituted aryl;
wherein B is hydrogen, phenyl, naphthyl, or a 5- to 6-membered heteroaryl ring, optionally fused to a benzo group or heteroaryl ring, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, (C₁-C₈) alkoxy, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷ wherein v is zero, one, two or three and —COR¹⁶ and R¹⁷ taken together with the nitrogen to which they are attached may form a 4- to 6-membered lactam ring.

In one aspect, the invention relates to compounds having the following formula, denoted herein as formula Ia: 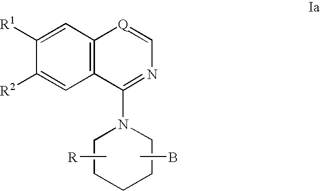
and to pharmaceutically acceptable salts, solvates and prodrugs thereof;
wherein Q is N or CH;
wherein R¹, R² and R⁵ are independently H, halogen, —CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹, R² and R⁵ are independently alkoxy, alkenyloxy or alkyl, R¹ and R² or R¹ and R⁵ may optionally be connected to form a 5 to 8 membered ring; and when R¹, R² and R⁵ are —NR³R⁴, R³ and R⁴ may optionally combine with the nitrogen in which they are attached to form a 5 to 8 membered ring;
wherein R is H, —COOR³, —CONR³R⁴, —COR⁴, —NR³R⁴, —NHCOR³, —OH, —HNCOOR³, —CN, —HNCONHR⁴ (C₁-C₆)alkyl or —O(C₂-C₆) alkyl;
wherein R³ and R⁴ are independently H, (C₁-C₆)alkyl, aryl or substituted aryl;
wherein B is hydrogen, phenyl, naphthyl, or a 5- to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥ^{NCOR}¹⁶R¹⁷ wherein v is zero, one, two or three and —COR¹⁶ and R¹⁷ taken together with the nitrogen to which they are attached form a 4- to 6-membered lactam ring.

In another aspect of the present invention B is phenyl, phenyl substituted by (C₁-C₅)alkoxy, (C₁-C₅)alkyl, trifluoroalkyl or (C₂-C₅)trifluoroalkoxy.

In another aspect of the present invention B is phenyl substituted with trifluoromethyl.

In another aspect of the present invention R is hydrogen, (C₁-C₅)alkoxy, —NR³R⁴, —HNCOOR3, or hydroxyl.

In other aspect of the present invention R¹ and R² are each independently (C₁-C₆)alkoxy.

In another aspect of the present invention R¹ and R² are each ethoxy or methoxy.

In another aspect of the present invention R¹ and R² are each independently (C₁-C₆)alkoxy, X and Z are N, Y is CH, B is phenyl or substituted phenyl and R is —NHCOR³.

In another aspect of the present invention R¹ and R² are each independently (C₁-C₆)alkoxy, Q is N, B is phenyl or substituted phenyl and R is —NHCOR³.

In another aspect of the present invention R¹ is methoxy when R² is ethoxy or R¹ is ethoxy when R² is methoxy.

In another aspect of the present invention, the heteroaryl group in substituent B is a heteroaryl or benzo-fused heteroaryl group selected from pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl

Examples of heteroaryl and benzo-fused heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

Specific examples of the compounds of the present invention are as follows:
N-[1-(6,7-Dimethoxy-quinazolin-4-yl)-3-phenyl-piperidin-4-yl]-benzamide;
N-[1-(6,7-Dimethoxy-quinazolin-4-yl)-3-pheny-piperidin-4-yl]-2,2-dimethyl-propionamide;
cis-1-(6,7-Dimethoxy-quinazolin-4-yl)-3-phenyl-piperidin-4-ol;
trans-1-(6,7-Dimethoxy-quinazolin-4-yl)-3-phenyl-piperidin-4-ol;
1′-(6,7-Dimethoxy-quinazolin-4-yl)-1′,2′,3′,4′,5′,6′-hexahydro-[2,3]bipyridinyl-4′-ol;
1-(6-Ethoxy-7-methoxy-quinazolin-4-yl)-5-phenyl-piperidin-3-ol;
1-(6,7-Dimethoxy-quinazolin-4-yl)-5-phenyl-piperidine;
7-Methoxy-4-(3-phenyl-piperidin-1-yl)-6-propoxy-quinazoline;
4-[3-(5-Fluoro-1H-benzoimidazol-2-yl)-piperidin-1-yl]-6,7-dimethoxy-quinazoline;
1-(6,7-Dimethoxy-quinazolin-4-yl)-5-phenyl-piperidin-3-ol;
trans-1-(6,7-Dimethoxy-quinazolin-4-yl)-5-phenyl-piperidin-3-ol;
4-(3-Benzooxazol-2-yl-piperidin-1-yl)-6,7-dimethoxy-quinazoline;
1-(6,7-Dimethoxy-quinazolin-4-yl)-5-phenyl-piperidin-3-ylamine hydrochloride;
1-(6,7-Dimethoxy-quinazolin-4-yl)-5-(4-methoxy-phenyl)-piperidin-3-ol;
6,7-Dimethoxy-4-[3-(5-phenyl-oxazol-2-yl)-piperidin-1-yl]-quinazoline;
6,7-Dimethoxy-4-[3-(4-methoxy-phenyl)-piperidin-1-yl]-quinazoline;
1-(6,7-Dimethoxy-quinazolin-4-yl)-3-phenyl-piperidin-3-ol;
cis-1-(6,7-Dimethoxy-quinazolin-4-yl)-5-naphthalen-1-yl-piperidin-3-ol;
6,7-Dimethoxy-4-[3-(3-methoxy-phenyl)-piperidin-1-yl]-quinazoline;
6,7-Dimethoxy-4-[3-(4-trifluoromethyl-phenyl)-piperidin-1-yl]-quinazoline;
6,7-Dimethoxy-4-[3-(5,6,7,8-tetrahydro-naphthalen-2-yl)-piperidin-1-yl]-quinazoline;
1-(6,7-Dimethoxy-quinazolin-4-yl)-4-phenyl-piperidine-4-carbonitrile;
1-(4-Methoxy-1,3-dioxa-7,9-diaza-cyclopenta[a]naphthalen-6-yl)-5-(4-methoxy-phenyl)-piperidin-3-ol;
1-(10-Methoxy-2,3-dihydro-1,4-dioxa-5,7-diaza-phenanthren-8-yl)-5-(4-methoxy-phenyl)-piperidin-3-ol;
[1-(10-Methoxy-2,3-dihydro-1,4-dioxa-5,7-diaza-phenanthren-8-yl)-5-(4-methoxy-phenyl)-piperidin-3-yl]-carbamic acid methyl ester;
5-(4-Methoxy-phenyl)-1-(6,7,8-trimethoxy-quinazolin-4-yl)-piperidin-3-ol;
[5-(4-Methoxy-phenyl)-1-(6,7,8-trimethoxy-quinazolin-4-yl)-piperidin-3-yl]-carbamic acid methyl ester;
1-(6,7-Dimethoxy-cinnolin-4-yl)-5-(4-methoxy-phenyl)-piperidin-3-ol; and
[1-(6,7-Dimethoxy-cinnolin-4-yl)-5-(4-methoxy-phenyl)-piperidin-3-yl]-carbamic acid methyl ester.

The above listed compounds and their pharmaceutically salts, solvates, and prodrugs thereof are preferred embodiments of the subject invention.

Compounds of Formula I may have optical centers and therefore may occur in different enantiomeric and diastereomeric configurations. The present invention includes all enantiomers, diastereomers, and other stereoisomers of such compounds of Formula I as well as racemic compounds and racemic mixtures and other mixtures of stereoisomers thereof.

This invention also pertains to a pharmaceutical composition for treatment of certain psychotic disorders and conditions such as schizophrenia, delusional disorders and drug induced psychosis; to anxiety disorders such as panic and obsessive-compulsive disorder; and to movement disorders including Parkinson's disease and Huntington's disease, comprising an amount of a compound of formula I effective in inhibiting PDE10.

In another embodiment, this invention relates to a pharmaceutical composition for treating psychotic disorders and condition such as schizophrenia, delusional disorders and drug induced psychosis; anxiety disorders such as panic and obsessive-compulsive disorder; and movement disorders including Parkinson's disease and Huntington's disease, comprising an amount of a compound of formula I effective in treating said disorder or condition.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Examples of movement disorders that can be treated according to the present invention include but are not limited to Huntington's disease and dyskinesia associated with dopamine agonist therapy, Parkinson's disease, restless leg syndrome, and essential tremor.

Other disorders that can be treated according to the present invention are obsessive/compulsive disorders, Tourette's syndrome and other tic disorders.

In another embodiment, this invention relates to a method for treating an anxiety disorder or condition in a mammal which method comprises administering to said mammal an amount of a compound of formula I effective in inhibiting PDE10.

This invention also provides a method for treating an anxiety disorder or condition in a mammal which method comprises administering to said mammal an amount of a compound of formula I effective in treating said disorder or condition.

Examples of anxiety disorders that can be treated according to the present invention include, but are not limited to, panic disorder; agoraphobia; a specific phobia; social phobia; obsessive-compulsive disorder; post-traumatic stress disorder; acute stress disorder; and generalized anxiety disorder.

This invention further provides a method of treating a drug addiction, for example an alcohol, amphetamine, cocaine, or opiate addiction, in a mammal, including a human, which method comprises administering to said mammal an amount of a compound of formula I effective in treating drug addiction.

This invention also provides a method of treating a drug addiction, for example an alcohol, amphetamine, cocaine, or opiate addiction, in a mammal, including a human, which method comprises administering to said mammal an amount of a compound of formula I effective in inhibiting PDE10.

A “drug addiction”, as used herein, means an abnormal desire for a drug and is generally characterized by motivational disturbances such a compulsion to take the desired drug and episodes of intense drug craving.

This invention further provides a method of treating a disorder comprising as a symptom a deficiency in attention and/or cognition in a mammal, including a human, which method comprises administering to said mammal an amount of a compound of formula I effective in treating said disorder.

This invention also provides a method of treating a disorder or condition comprising as a symptom a deficiency in attention and/or cognition in a mammal, including a human, which method comprises administering to said mammal an amount of a compound of formula I effective in inhibiting PDE10.

This invention also provides a method of treating a disorder or condition comprising as a symptom a deficiency in attention and/or cognition in a mammal, including a human, which method comprises administering to said mammal an amount of a compound of formula 1 effective in treating said disorder or condition.

The phrase “deficiency in attention and/or cognition” as used herein in “disorder comprising as a symptom a deficiency in attention and/or cognition” refers to a subnormal functioning in one or more cognitive aspects such as memory, intellect, or learning and logic ability, in a particular individual relative to other individuals within the same general age population. “Deficiency in attention and/or cognition” also refers to a reduction in any particular individual's functioning in one or more cognitive aspects, for example as occurs in age-related cognitive decline.

Examples of disorders that comprise as a symptom a deficiency in attention and/or cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; and age-related cognitive decline.

This invention also provides a method of treating a mood disorder or mood episode in a mammal, including a human, comprising administering to said mammal an amount of a compound of formula I effective in treating said disorder or episode.

This invention also provides a method of treating a mood disorder or mood episode in a mammal, including a human, comprising administering to said mammal an amount of a compound of formula I effective in inhibiting PDE10.

Examples of mood disorders and mood episodes that can be treated according to the present invention include, but are not limited to, major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder.

This invention further provides a method of treating a neurodegenerative disorder or condition in a mammal, including a human, which method comprises administering to said mammal an amount of a compound of formula I effective in treating said disorder or condition.

This invention further provides a method of treating a neurodegenerative disorder or condition in a mammal, including a human, which method comprises administering to said mammal an amount of a compound of formula I effective in inhibiting PDE10.

As used herein, and unless otherwise indicated, a “neurodegenerative disorder or condition” refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons. The term “neurotrophic agent” as used herein refers to a substance or agent that has some or all of these properties.

Examples of neurodegenerative disorders and conditions that can be treated according to the present invention include, but are not limited to, Parkinson's disease; Huntington's disease; dementia, for example Alzheimer's disease, multi-infarct dementia, AIDS-related dementia, and Fronto temperal Dementia; neurodegeneration associated with cerebral trauma; neurodegeneration associated with stroke, neurodegeneration associated with cerebral infarct; hypoglycemia-induced neurodegeneration; neurodegeneration associated with epileptic seizure; neurodegeneration associated with neurotoxin poisoning; and multi-system atrophy.

In one embodiment of the present invention, the neurodegenerative disorder or condition comprises neurodegeneration of striatal medium spiny neurons in a mammal, including a human.

In a further embodiment of the present invention, the neurodegenerative disorder or condition is Huntington's disease.

The term “aryl”, as used herein, unless otherwise indicated, includes an organic radical derived from a univalent aromatic hydrocarbon and includes but is not limited to, phenyl, naphthyl and indenyl.

The term “alkyl”, as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “alkenyl”, as used herein, unless otherwise indicated, includes alkyl moieties having at least one carbon-carbon double bond wherein alkyl is as defined above. Examples of alkenyl include, but are not limited to, ethenyl and propenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl.

The term “cycloalkyl”, as used herein, unless otherwise indicated, includes alkyl groups comprising non-aromatic saturated cyclic alkyl moieties wherein alkyl is as defined above. Examples of cycloalkyl include, but are not limited to, cyclopropyl, cyclopropylethyl, cyclopropylmethyl, cyclobutyl, cyclopentyl, cyclohexyl, and cycloheptyl.

“Heteroaryl”, as used herein, refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

“Neurotoxin poisoning” refers to poisoning caused by a neurotoxin. A neurotoxin is any chemical or substance that can cause neural death and thus neurological damage. An example of a neurotoxin is alcohol, which, when abused by a pregnant female, can result in alcohol poisoning and neurological damage known as Fetal Alcohol Syndrome in a newborn. Other examples of neurotoxins include, but are not limited to, kainic acid, domoic acid, and acromelic acid; certain pesticides, such as DDT; certain insecticides, such as organophosphates; volatile organic solvents such as hexacarbons (e.g. toluene); heavy metals (e.g. lead, mercury, arsenic, and phosphorous); aluminum; certain chemicals used as weapons, such as Agent Orange and Nerve Gas; and neurotoxic antineoplastic agents.

As used herein, the term “selective PDE10 inhibitor” refers to a substance, for example an organic molecule that effectively inhibits an enzyme from the PDE10 family to a greater extent than enzymes from the PDE 1-9 families or PDE11 family. In one embodiment, a selective PDE10 inhibitor is a substance, for example an organic molecule, having a Kᵢ for inhibition of PDE10 that is less than or about one-tenth the Kᵢ that the substance has for inhibition of any other PDE enzyme. In other words, the substance inhibits PDE10 activity to the same degree at a concentration of about one-tenth or less than the concentration required for any other PDE enzyme.

The term “provided that at least one of X, Y and Z must be N or CH” means that X, Y and Z cannot simultaneously be all N or CH. At least one of X, Y and Z must be N and at least one of X, Y and Z must be CH.

In general, a substance is considered to effectively inhibit PDE10 activity if it has a K of less than or about 10 μM, preferably less than or about 0.1 μM.

A “selective PDE10 inhibitor” can be identified, for example, by comparing the ability of a substance to inhibit PDE10 activity to its ability to inhibit PDE enzymes from the other PDE families. For example, a substance may be assayed for its ability to inhibit PDE10 activity, as well as PDE1, PDE2, PDE3A, PDE4A, PDE4B, PDE4C, PDE4D, PDE5, PDE6, PDE7, PDE8, PDE9, PDE11 and so-on.

The term “treating”, as in “a method of treating a disorder”, refers to reversing, alleviating, or inhibiting the progress of the disorder to which such term applies, or one or more symptoms of the disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffecctive disorders include disorganized speech, affective flattening, alogia, anhedonia, inappropriate affect, dysphoric mood (in the form of, for example, depression, anxiety or anger), and some indications of cognitive dysfunction.

The term “mammal”, as used herein, refers to any member of the class “Mammalia”, including, but not limited to, humans, dogs, and cats.

Compounds of Formula I containing one or more asymmetric carbon atoms can exist as two or more stereoisomers. Where a compound of Formula I contains an alkenyl or alkenylene group, geometric cisitrans (or Z/E) isomers are possible. Where structural isomers are interconvertible via a low energy barrier, tautomeric isomerism (‘tautomerism’) can occur. This can take the form of proton tautomerism in compounds of Formula I containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate or racemic mixture (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

This invention also pertains to an intermediate compound of formula II and its derivatives which are used in the preparation of compounds of formula I 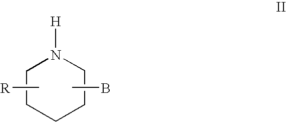
wherein R is H, —COOR³, —CONR³R⁴, —COR⁴, —NR³R⁴, —NCOR³, —OH, —HNCOOR³, —CN, —HNCONHR⁴ (C₁-C₆)alkyl, (C₂-C₆) alkoxy or (C₂-C₆)trifluoroalkoxy;
wherein R³ and R⁴ are independently H, (C₁-C₆) alkyl, aryl or substituted aryl.
wherein B is hydrogen, phenyl, naphthyl, or a 5- to 6-membered heteroaryl ring, optionally fused to a benzo group, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from halo-, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷ wherein v is zero, one, two or three and —COR¹⁶ and R¹⁷ taken together with the nitrogen to which they are attached form a 4- to 6-membered lactam ring.

In another embodiment the present invention relates to a process for preparing a compound of the formula 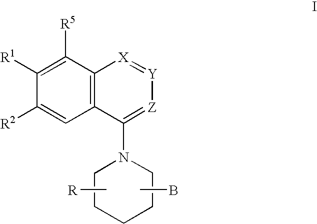
or a pharmaceutically acceptable salt, solvate or prodrug thereof,
wherein X, Y and Z are each independently N or CH, provided that at least one of X, Y and Z must be N or CH and provided that when Z is nitrogen, Y is CH; and when Y is nitrogen, X is nitrogen and Z is CH;
wherein R¹, R² and R⁵ are independently H, halogen, —CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹, R² and R⁵ are independently alkoxy, alkenyloxy or alkyl, R¹ and R² or R¹ and R⁵ may optionally be connected to form a 5 to 8 membered ring; and when R¹, R² and R⁵ are —NR³R⁴, R³ and R⁴ may optionally combine with the nitrogen in which they are attached to form a 5 to 8 membered ring;
wherein R⁴ is H, —COOR³, —CONR³R⁴, —COR⁴, —NR³R⁴, —NHCOR³, —OH, —HNCOOR³, —CN, —HNCONHR⁴, (C₁-C₆)alkyl or (C₂-C₆) alkoxy;
wherein R³ and R⁴ are independently H, (C₁-C₆) alkyl, alkenyl, aryl or substituted aryl;
wherein B is hydrogen, phenyl, naphthyl, or a 5- to 6-membered heteroaryl ring, optionally fused to a benzo group, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, (C₁-C₈) alkoxy, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷ wherein v is zero, one, two or three and —COR¹⁶ and R¹⁷ taken together with the nitrogen to which they are attached may form a 4- to 6-membered lactam ring.
comprising reacting a compound of formula IIa 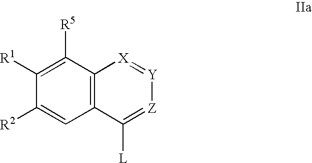
wherein L is a suitable leaving group;
with a compound of formula II 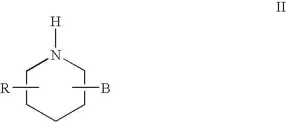
   wherein R¹, R², R⁵, X, Y, Z, R and B are defined above.

In another embodiment L is a leaving group comprising a halogen atom selected from chlorine, bromine and iodine.

In another embodiment, the compound is preferably produced in the presence of a base.

In another embodiment the present invention relates to a process for preparing a compound of formula I 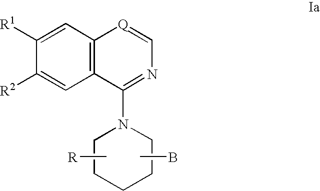
and to pharmaceutically acceptable salts, solvates and prodrugs thereof,
wherein Q is N or CH;
wherein R¹ and R² are independently H, halogen, CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹ and R² are independently alkoxy, alkenyloxy or alkyl, R¹ and R² may optionally be connected to form a 5 to 8 membered ring; and when R¹ and R² are —NR³R⁴, R³ and R⁴ may optionally combine with the nitrogen in which they are attached to form a 5 to 8 membered ring;
wherein R is H, —COOR³, —CONR³R⁴, —COR⁴, —NR³R⁴, —OH, —HNCOOR³, —CN, —HNCONHR⁴ (C₁-C₆)alkyl or —O(C₂-C₆) alkyl;
wherein R³ and R⁴ are independently H(C₁-C₆) alkyl, aryl or substituted aryl.
wherein B is hydrogen, phenyl, naphthyl, or a 5- to 6-membered heteroaryl ring, optionally fused to a benzo group, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from halo-, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷ wherein v is zero, one, two or three and —COR¹⁶ and R¹⁷ taken together with the nitrogen to comprising reacting a compound of formula III 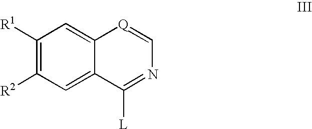
   Q is N or CH;
wherein R¹ and R² are independently H halogen, CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹ and R² are independently alkoxy, alkenyloxy or alkyl, R¹ and R² may optionally be connected to form a 5 to 8 membered ring; and when R¹ and R² are —NR³R⁴, R³ and R⁴ may optionally combine with the nitrogen in which they are attached to form a 5 to 8 membered ring;
and L is a suitable leaving group; with a compound of formula II 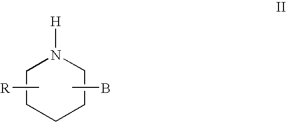
   wherein R and B are defined above,
preferably in the presence of a base.

Examples of leaving groups for the above processes include, but are not limited to chlorine, bromine, iodine, p-toluenesulfonate, alkyl sulfate and alkanesulfonate, particularly trifluoromethanesulfonate

In a preferred embodiment, the leaving group L is chlorine.

### DETAILED DESCRIPTION OF THE INVENTION

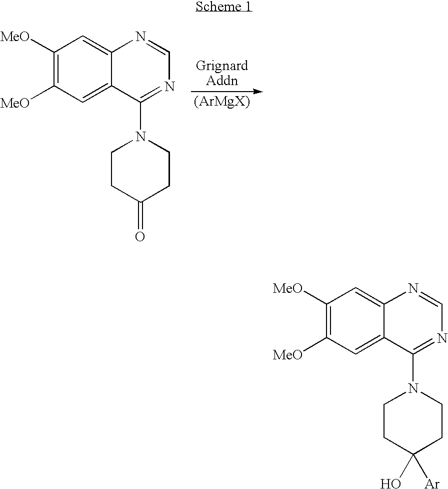

Scheme 1 shows a method for preparing quinazoline compounds substituted in the 4-position with (4-hydroxy-4-aryl)-piperidine derivatives. The method begins with 1-(6,7-dimethoxy-quinazolin-4-yl)-piperidin-4-one, which is prepared according to a method similar to Scheme 5. Treatment with Grignard reagents according to well-known procedures provides the target compounds.

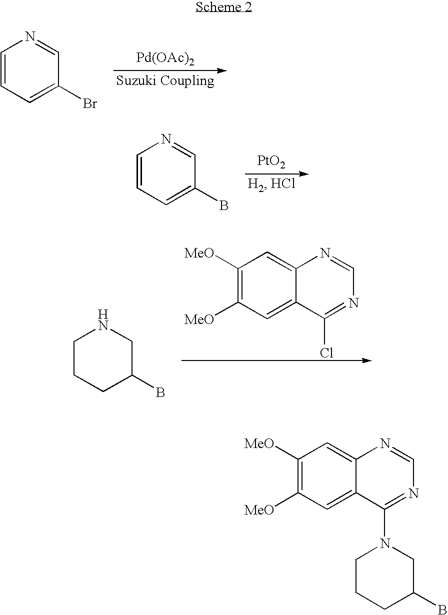

Scheme 2 depicts a synthetic route to 6,7-Dimethoxy-4-(3-aryl-piperidin-1-yl)-quinazoline. The route begins with 3-bromopyridine. The desired 3-aryl group can be installed via the well-known Suzuki coupling reaction utilizing any of the many conditions reported in the literature [Miyaura, N. and A. Suzuki, Palladium-catalyzed cross-coupling reactions of organoborane compounds. Chem. Rev., 1995. 95: p. 2457-2483.] A preferred set of conditions for reduction of the pyridine ring to the piperidine involves hydrogenation in the presence of a catalyst such as platinum oxide. The resultant substituted piperidine is coupled with the desired substituted 4-chloroquinazoline via the method described in Scheme 5.

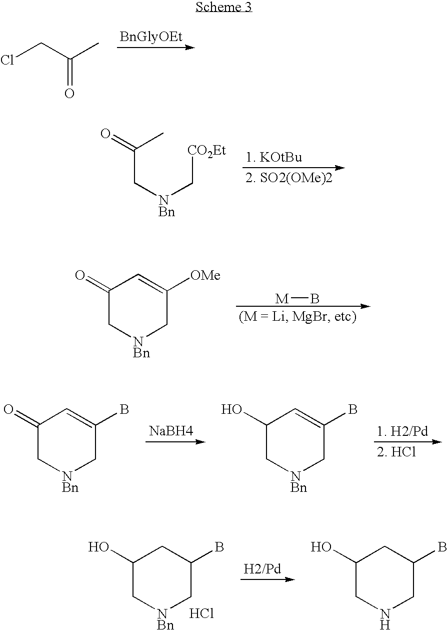

Scheme 3 shows a published method [GB2060617A, R. G. Shepherd & A. C. White] for the preparation of 3-hydroxy-5-arylpiperidines. The final product piperidines can be coupled with 4-chloroquinazolines as in Scheme 5.

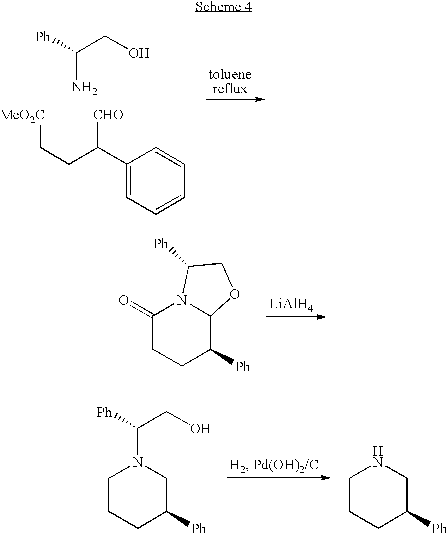

Scheme 4 describes a published method [Amat, M. et al. J. Org. Chem. 2002, 67, 5343-5351] for the synthesis of optically active 3-phenylpiperidines. The product piperidine can be coupled with a 4-chloroquinazoline derivative according to the method of Scheme 5.

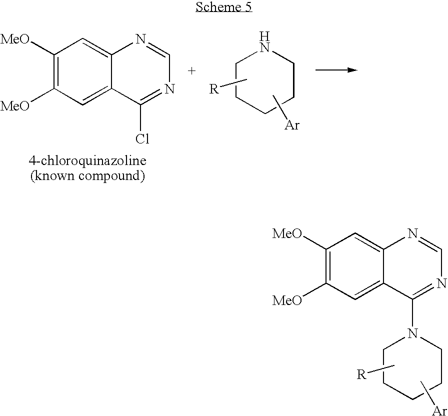

Scheme 5 depicts a coupling reaction between 4-chloro-6,7-dimethoxyquinazoline [PC Int. Appl. 2003008388, 30 January 2003; Wright, S. W., et al., Anilinoquinazoline inhibitors of fructose 1,6-biphosphatase bind at a novel allosteric site: synthesis, in vitro characterization, and x-ray crystallography. J. Med. Chem., 2002. 45: p. 3865-3877] and a piperidine component to generate the desired product. This reaction is not limited to 4-chloro-6,7-dimethoxyquinazoline, since other substituted 4-chloroquinazolines undergo this reaction in similar fashion. This reaction is typically carried out in an inert solvent such as toluene, with or without the addition of a base, at temperatures ranging from about 0° C. to 200° C. Microwave irradiation may also be used to facilitate the reaction. Other suitable solvents include but are not limited to ether, THF, benzene, chloroform, dioxane, ethyl acetate, 2-propanol, water and xylene. Alternatively, solvent mixtures such as toluene/isopropanol or THF/water can be used. A preferred set of conditions includes treatment of the chloro-quinazoline component and the substituted piperidine component in toluene/isopropanol at reflux for 2-24 hours. Another preferred set of conditions involves treatment of the chloro-quinazoline component and the substituted piperidine component in THF/saturated sodium bicarbonate at 60° C. for 2-24 hours.

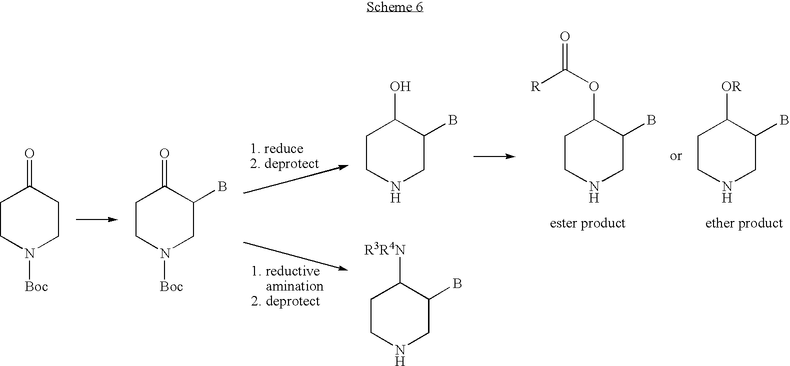

Scheme 6 depicts a method for the preparation of 3-aryl piperidine derivatives with nitrogen or oxygen based substitution at the 4-position. The sequence shown is illustrated with 4-oxo-piperidine-1-carboxylic acid tert-butyl ester (N-Boc-4-oxo-piperidine), but other carbamate protection can be used in place of the Boc-group. Examples include the Cbz or Fmoc groups. The protecting functionality is not limited to carbamate groups, as amide protection or alkyl protection can be used as well. Examples of amide protection include the acetyl and trifluoroactyl groups. Examples of the alkyl protecting groups include the benzyl group, or the paramethoxy-benzyl group. The 3-aryl group is incorporated via a palladium catalyzed arylation reaction utilizing the desired aryl chloride or aryl bromide. A large range of catalysts, solvents and conditions may be used for this conversion. For example, the possible solvents include but are not limited to THF, ether, dioxane, glyme, DMF, toluene, benzene or Xylene, or mixtures thereof. Possible palladium catalysts include, but are not limited to, Pd (PPh₃)₄, Pd₂(dba)₃, or Pd(dppf)Cl₂. The palladium catalysts can be purchased or prepared in situ. Possible bases include, but are not limited to, Cs₂CO₃, CsF, K₃PO₄, KF, Na₂CO₃, and K₂CO₃. One example set of conditions involves heating the piperidine, palladium acetate, sodium tert-butoxide, tri-tert butylphosphine, and the desired aryl bromide in THF. A range of other conditions is possible, and many are described in the literature. [Culkin, D. A. and J. F. Hartwig, Palladium-Catalyzed (x-Arylation of Carbonyl Compounds and Nitriles. Acc. Chem. Res., 2003. 36: p. 234-245 and Fu, G. C. and A. F. Littke, Angew. Chem. Int. Ed., 2002. 41: p. 4176-4211.]

After incorporating the aryl group, the carbonyl group is reduced to a hydroxyl group utilizing any of the many known methods. Most commonly, this is done by treatment with a borohydride reagent in an inert solvent. Sodium borohydride, lithium borohydride, or sodium cyanoborohydride in THF or ether are often used. The resultant alcohol may be utilized without further modification of the hydroxyl group. Alternatively, it may be alkylated to form an ether, or acylated to form an ester. In each case, the protecting group is then removed via standard conditions according to methods commonly known and available in the literature [Greene, T. W. and P. G. M. Wuts, Protective Groups in Organic Synthesis. 1999, New York: John Wiley & Sons and Kocienski, P. J., Protecting Groups. 1994, New York: Georg Thieme Verlag Stuttgart.] Subsequent to Boc removal, the derivatized piperidine is coupled with the desired 4-chloroquinazoline compound according to the method described in Scheme 5. A nitrogen atom or nitrogen-containing group such as carbamate, amide, urea, or heterocycle may replace the 4-hydroxyl group. This may be done subsequent to coupling with the quinazoline, but preferably it is done prior. This is accomplished starting with the product of the arylation reaction. The ketone group is converted into an amine group by utilizing the well-known reductive amination reaction. In this reaction, ammonia or a primary or secondary amine is treated with the ketone and a reducing agent in a suitable solvent. There are many effective reducing agents known to those skilled in the art. Two of the most common reducing agents are sodium cyanoborohydride and sodium triacetoxyborohydride. However, other less common reducing agents can be used. Catalytic hydrogenation is another alternative. Suitable solvents include various alcohols, as well as inert solvents such as methylene chloride, THF, ether, toluene, ethyl acetate, benzene, glyme, or chloroform. Preferably, alcoholic solvents are used with sodium cyanoborohydride and catalytic hydrogenation, while the inert solvents are often used with sodium triacetoxyborohydride. The product of the reaction can be deprotected and coupled with the quinazoline as described above. However, when the amine source for the reductive amination reaction is either ammonia or a primary amine, the reaction product can be further modified by alkylation or acylation. Both reactions are well-known to those skilled in the art, and methods are readily available in the chemical literature [Bodanszky, M., Principles of Peptide Synthesis. 2nd ed. 1993, Berlin Heidelberg: Springer-Verlag, Humphrey, J. M. and A. R. Chamberlin, Chemical Synthesis of Natural Product Peptides: Coupling Methods for the Incorporation of Noncoded Amino Acids into Peptides. Chem. Rev., 1997. 97(6): p. 2243-2266 and Furness, B. S., et al., Vogel's Textbook of Practical Organic Chemistry. 5 ed. 1989: Prentice Hall.] Subsequent to alkylation or acylation, the product is deprotected and coupled with the quinazoline as described above.

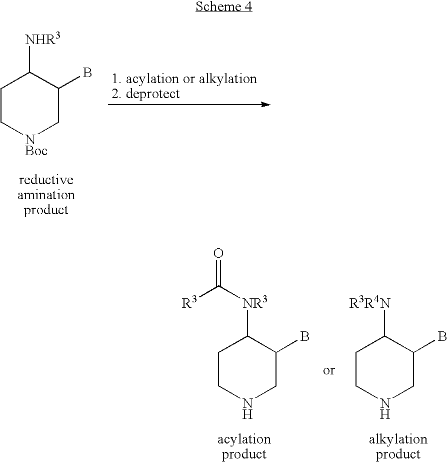

Scheme 7 shows a method for treating the reductive amination product of Scheme 8 to provide 3-aryl-4-acylamino- or 3-aryl-4-dialkylamino-piperidines. The sequence is illustrated utilizing Boc protection of the piperidine nitrogen atom, but other carbamate or acyl protection can be used. Common examples include Cbz or trifluoroacetate protection. After the desired alkylation or acylation via standard protocols, the product piperidines can be deprotected and coupled with a 4-chloroquinazoline as described in Scheme 5.

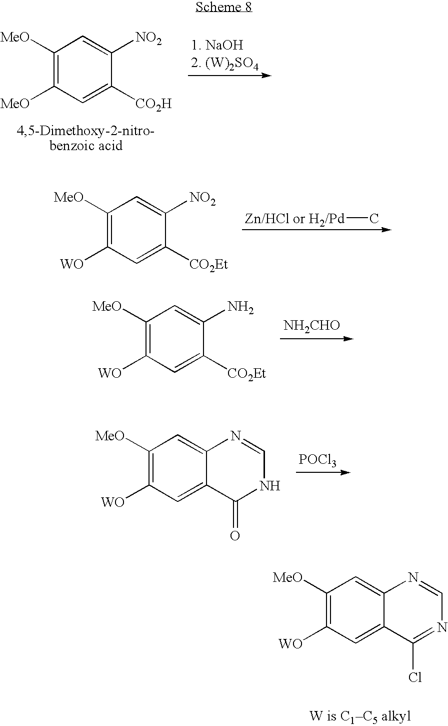

Scheme 8 shows a sequence for the synthesis of quinazoline intermediates in which the alkoxy groups in the 6- and 7-positions are different. According to one method, 4,5-dimethoxy-2-nitro-benzoic acid selectively demethylated with sodium hydroxide to give a new benzoic acid derivative. Alkylation with dialkyl sulfate or an alkyl iodide provides the new substituted benzene in which the alkoxy groups are different. Zinc reduction of the nitro group to an aniline is followed by sequential reaction with formamide and phosphorous oxychloride to provide a 4-chloroquinazoline compound possessing a methoxy group in the 7-position and a different alkoxy group in the 6-position. This quinazoline can be coupled with amines via the method described in Scheme 7.

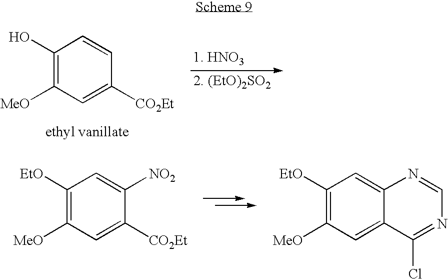

Scheme 9 shows a related method that allows for the alternative substitution pattern. In this sequence, commercially available ethylvanillate is nitrated with nitric acid, and then alkylated with the desired electrophile. For example, diethylsulfate or iodoethane can be used to install an ethyl group as shown. di-n-propyl sulfate would be used to install a propyl group, and so on. Zinc reduction and conversion into the 4-chloroquinazoline occurs as in Scheme 10, but the product in this case possesses a methoxy group in the quinazoline 6-position, and a different alkyloxy group resides in the 7-position. Catalytic hydrogenation may also be used to reduce the nitro group.

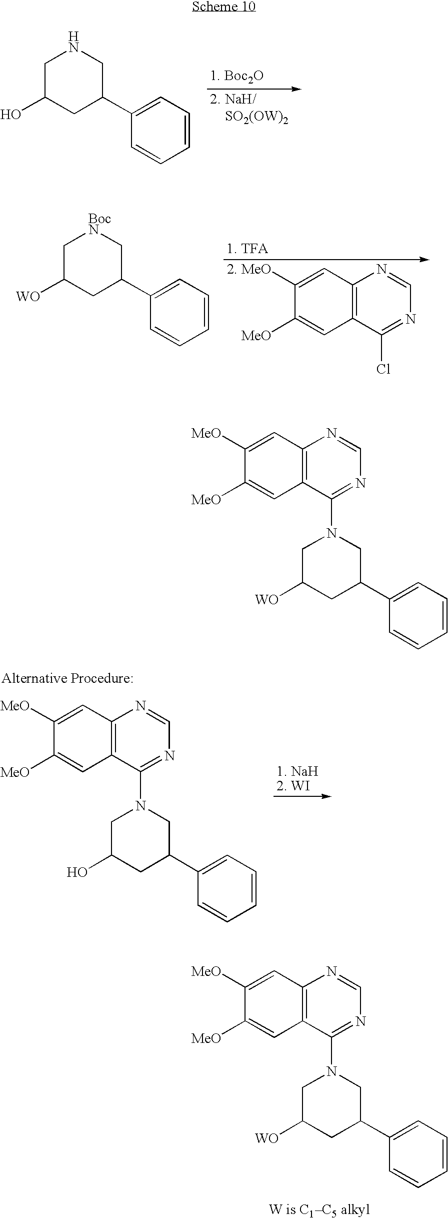

Scheme 10 depicts a method for incorporating an alkoxy group into the 3-position of the piperidine ring. The method begins with the 3-hydroxyl-5-aryl piperidine (prepared via Scheme 3), which is first protected on nitrogen with a suitable carbamate protecting group such as the Boc group using standard methods. This is followed by alkylation, which is preferably accomplished by generation of the alkoxide with a strong base such as sodium hydride, LDA, or LHMDS in an inert solvent such as THF or ether or DMF at temperatures ranging from 0° C. to room temperature. The alkoxide is then treated with an alkylating agent such as a dialkylsulfoxide or an alkyl halide. The resultant ether is easily deprotected under acidic conditions, such as with trifluoroacetic acid, and then coupled with the chloroquinazoline utilizing methods described herein. Alternatively, also shown in Scheme 10, the piperidine can first be coupled with the chloroquinazoline via the Scheme 5 procedure. The coupled product can then be treated with sodium hydride followed by the desired dialkylsulfate or alkyl halide to generate the ether product.

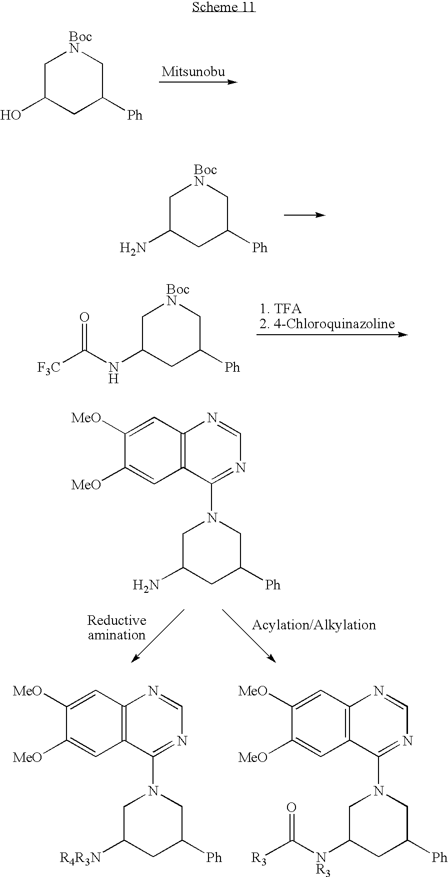

Scheme 11 depicts a method used for the preparation of 4-piperidylpiperidines possessing 3-amino or amido functionality on the piperidine ring. The method begins with the N-Boc-3-hydroxy-5-arylpiperidine shown, which is prepared via procedures shown herein. The Mitsunobu reaction is used to install the amino group [Fabiano, E., B. T. Golding, and M. M. Sadeghi, A simple conversion of alcohols into amines. Synthesis, 1987: p. 190-192.] Alternatively, the amine can be accessed from the corresponding carboxylic acid precursor via the curtius rearrangement. The amine must then be protected prior to coupling with the 4-chloroquinazoline. This can be accomplished via protection as the trifluoroacetyl group (as shown) although other protecting groups may be used as well. After Boc cleavage with acid and incorporation of the quinazoline group, reductive alkylations or acylations can be used to incorporate the desired groups. These methods are described above.

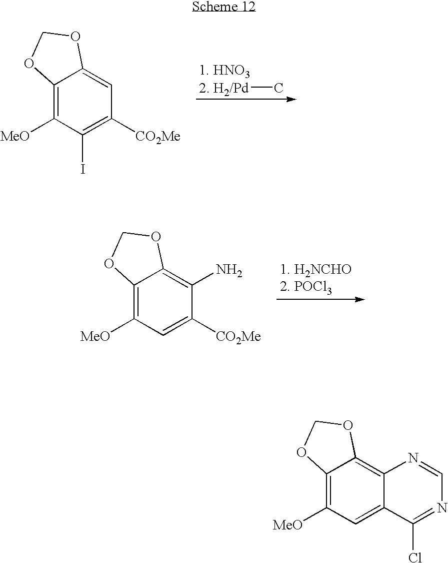

Scheme 12 illustrates how the dioxolane structure was incorporated into the quinazoline ring in the formation of 6-Chloro-4-methoxy-1,3-dioxa-7,9-diaza-cyclopenta[a]naphthalene. The method begins with the 3,4-methylenedioxy aryl iodide obtained according to the literature procedure in Chang, J., et al., Efficient Synthesis of g-DDB. Bioorg. Med. Chem. Lett., 2004. 14: p. 2131-2136. The compound undergoes a nitration reaction mediated by nitric acid or copper nitrate at the open aryl site, and a subsequent palladium catalyzed hydrogenation is utilized to cleave the iodide and reduce the nitro group to the amino group. The resultant anthranilic acid derivative is converted into the 4-chloroquinazoline derivative by sequential treatment with formamide and phosphorous oxy chloride according the Scheme 8 methods. Coupling of the quinazoline with amine nucleophiles proceeds according to conditions described in Scheme 5.

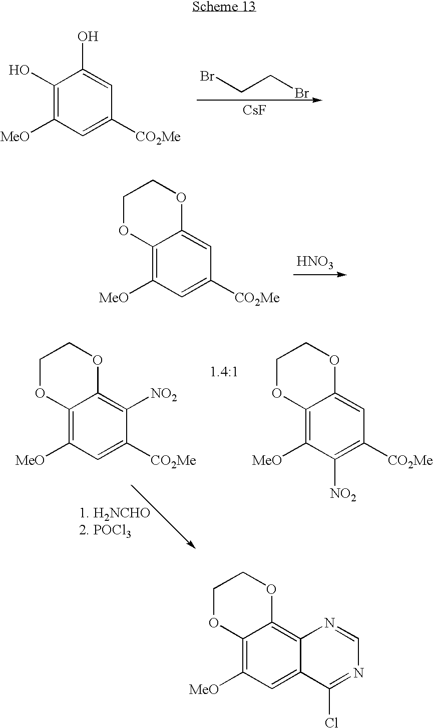

Scheme 13 describes how the dioxane ring is incorporated into the quinazoline ring system. According to this method, methyl-3,4-dihydroxy-5-methoxybenzoate was alkylated with 1,2-dibromoethane in dimethylformamide in the presence of CsF. The resultant dioxane derivative is nitrated with nitric acid in the usual way to give a ˜1.4:1 mixture of two nitrated compounds. Of these, the major isomer is isolated by chromatography and used to form the 4-chloroquinazoline using the dimethylformamide/POCl3 methods described above. Coupling with amine nucleophiles likewise occurs as in Scheme 5 above to give the 4-aminoderivatives.

The acids which are used to prepare the pharmaceutically acceptable acid addition salts of the base compounds of this invention are those which form non-toxic acid addition salts, e.g. salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate, i.e., 1,1′-methylene-bis-(2-hydroxy-3-naphthoate), salts.

The compound of the invention may be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration, or in a form suitable for administration by inhalation or insufflation.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration, the composition may take the form of tablets or lozenges formulated in conventional manner.

The compounds of the invention may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogen-free water, before use.

When a product solution is required, it can be made by dissolving the isolated inclusion complex in water (or other aqueous medium) in an amount sufficient to generate a solution of the required strength for oral or parenteral administration to patients. The compounds may be formulated for fast dispersing dosage forms (fddf), which are designed to release the active ingredient in the oral cavity. These have often been formulated using rapidly soluble gelatin-based matrices. These dosage forms are well known and can be used to deliver a wide range of drugs. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect.

The compounds of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insulator may be formulated containing a powder mix of a compound of the invention and a suitable powder base such as lactose or starch.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

A proposed daily dose of the compound of the invention for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

Assay methods are available to screen a substance for inhibition of cyclic nucleotide hydrolysis by the PDE10 and the PDEs from other gene families. The cyclic nucleotide substrate concentration used in the assay is ⅓ of the Kₘ concentration, allowing for comparisons of IC₅₀ values across the different enzymes. PDE activity is measured using a Scintillation Proximity Assay (SPA)-based method as previously described (Fawcett et al., 2000). The effect of PDE inhibitors is determined by assaying a fixed amount of enzyme (PDEs 1-11) in the presence of varying substance concentrations and low substrate, such that the IC₅₀ approximates the Kᵢ (cGMP or cAMP in a 3:1 ratio unlabelled to [³H]-labeled at a concentration of ⅓ Km). The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (Amersham) (containing 3 mM of the respective unlabelled cyclic nucleotide for PDEs 9 and 11). Plates are re-sealed and shaken for 20 min, after which the beads were allowed to settle for 30 minutes in the dark and then counted on a TopCount plate reader (Packard, Meriden, Conn.) Radioactivity units can be converted to percent activity of an uninhibited control (100%), plotted against inhibitor concentration and inhibitor IC₅₀ values can be obtained using the “Fit Curve’ Microsoft Excel extension.

Using such assay, compounds of the present invention were determined to have an IC₅₀ for inhibiting PDE10 activity of less than about 10 micromolar.

The following Examples illustrate the present invention. It is to be understood, however, that the invention, as fully described herein and as recited in the claims, is not intended to be limited by the details of the following Examples.

### EXPERIMENTAL PROCEDURES

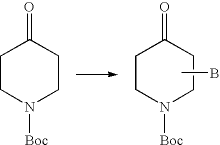

General Procedure 1 (alpha-arylation): A 1-L, three-neck, round bottom flask equipped with a magnetic stirrer and thermometer is purged with nitrogen and THF. Palladium acetate (0.05 mol %) and sodium tert-butoxide (1.5 mol %) is added and the mixture is stirred for 15 min to dissolve the butoxide base. Tri-tert butylphosphine (0.1 mol %), and the desired aryl halide derivative (1.1 mol %) are added, followed by 1-tert-butoxycarbonyl-4-piperidone (1.0 mol %). The reaction is heated at 45-50° C. over a period of 4 hr and the reaction mixture is then poured into a solution of sodium bicarbonate (15.0 g) in water (500 mL) and extracted with EtOAc (800 mL). The organic layer is dried and concentrated under reduced vacuum. Purification is accomplished via chromatography or crystallization. (B is as defined above)

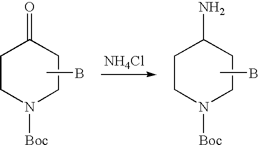

General procedure 2 (amination): to the N-protected-3-aryl-oxopiperidine (1 mol %) in methanol was added anhydrous ammonium chloride (20 mol %) and 4 A molecular sieves (ca. 1 g/mol substrate). After stirring for 1 hour (1 h), sodium cyanoborohydride (0.6 mol %) is added and the mixture is stirred for 1 h. The mixture is filtered and the filtrate is concentrated under reduced pressure. The residue is then dissolved in ethyl acetate, washed sequentially with water and brine, dried with sodium sulfate and concentrated. If necessary, purification is accomplished via silica gel chromatography.

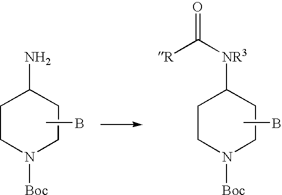

General procedure 3 (acvlation): To the 4-amino-N-Boc piperidine derivative (1.0 mol %) in methylene chloride is added the desired carboxylic acid (1.2 mol %), diisoopropylethylamine (5.0 mol %), and BOP (1.0 mol %). The mixture is stirred at room temperature (rt) for 4-12 h, at which point the solvent is removed under vacuum. The residue is dissolved in ethyl acetate and washed twice with water, once with brine, dried with magnesium sulfate, and concentrated. Purification is accomplished via silica gel chromatography or crystallization.

General procedure 4 (alternative acylation procedure): To the 4-amino-N-Boc piperidine derivative (1.05 mol %) in methylene chloride is added the desired carboxylic acid (1.1 mol %), triethylamine (2.0 mol %), and 1-propanephosphinic acid cyclic anhydride (PPACA, 1.1 mol %). The mixture is stirred at rt for 20 h, and then washed with 1 M sodium hydroxide, dried via filtration through cotton, and concentrated. Purification can be accomplished via chromatography if necessary

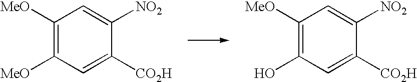

Preparation 1. 5-Hydroxy-4-methoxy-2-nitro-benzoic acid. To 4,5-Dimethoxy-2-nitro-benzoic acid was added 6 M NaOH (60 mL). The resultant yellow mixture was heated to 100° C. for 3 h, and then cooled to rt. The resultant solid was dissolved in 100 mL of water and poured into a slurry of 9 M HCl and crushed ice. The mixture was extracted twice with ethyl acetate and the combined extracts were washed with brine, dried over magnesium sulfate, and concentrated to give 14.7 g of a pale yellow solid. Recrystallization from ethyl acetate/hexanes provided 10.8 g (79%) of the title compound.

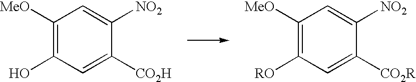

General Procedure 5. 4-Methoxy-2-nitro-5-alkoxy-benzoic acid alkyl ester. To 5-hydroxy-4-methoxy-2-nitro-benzoic acid in DMF (2.0 mL) is added 2.0 molar equivalents of potassium carbonate and 2.1 molar equivalents of the desired dialkylsulfoxide. The mixture is stirred at 85° C. for 8 h, cooled to rt, diluted with water, and extracted twice with ethyl acetate. The extracts are washed sequentially with 1 N NaOH and brine, dried with magnesium sulfate, and concentrated to give the title compound.

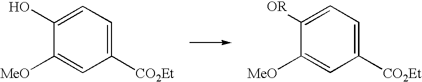

General Procedure 6. 4-Alkoxy-3-methoxy-benzoic acid ethyl ester. To ethyl vanillate and an excess of potassium carbonate in DMF is added 1.2 molar equivalents of the desired dialkylsulfate. The mixture is stirred for 24 h at room temperature, and then diluted with water and extracted with ether. The combined extracts are washed with brine, dried, and concentrated to give the title compound.

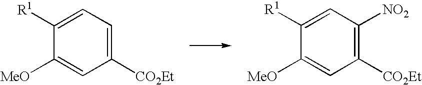

General Procedure 7. 4,5-dilkoxy-5-methoxy-2-nitro-benzoic acid ethyl ester. To the desired 3,4-dialkoxy-benzoic acid ethyl ester (ca. 10.0 g) in 12 mL of sulfuric acid at 0° C. is added dropwise 8 mL of a 1:1 mixture of sulfuric and nitric acids at such a rate to maintain the reaction temperature below 150C. The mixture is stirred at rt for 1 h and then poured into 100 g of crushed ice. The resultant aqueous mixture is extracted 3× with ethyl acetate and the combined extracts are washed with brine, dried with magnesium sulfate and concentrated. Silica gel chromatography eluting with hexane/ethyl acetate provides the title compound as a yellow solid.

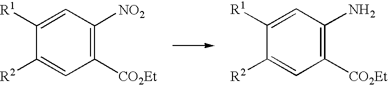

General Procedure 8. 2-Amino-4,5-dialkoxy-benzoic acid ethyl ester. To a slurry of the desired 4-alkoxy-5-methoxy-2-nitro-benzoic acid ethyl ester in 6 M HCl in an ice bath is added in portions an excess of zinc powder while maintaining the reaction temperature below 25° C. When TLC analysis indicates full consumption of starting material the mixture is diluted with cold water and extracted 3× with chloroform. The combined extracts are washed with brine and concentrated to provide the title compound as a white solid.

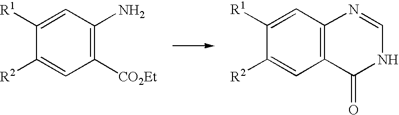

General Procedure 9. 6,7-dialkoxy-3H-quinazolin-4-one. To the 2-amino-4,5-dialkoxy-benzoic acid ethyl ester in formamide is added an excess of ammonium carbonate. The mixture is heated to 170° C. for 24 h, and then cooled to rt and poured into water. The resultant precipitate is collected via filtration. Silica gel chromatography eluting with hexane/ethyl acetate provides the title compound.

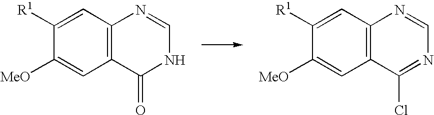

General Procedure 10. 4-Chloro-6,7-dialkoxy-quinazoline. A sample of 6,7-dialkoxyoxy-3H-quinazolin-4-one in POCl₃ is refluxed for 2 h. and then poured into a warm mixture of saturated aqueous NaHCO₃ and ethyl acetate. The mixture is stirred vigorously for 2 hr and the layers are separated. The organic portion is washed with brine, dried with magnesium sulfate and concentrated. Silica gel chromatography eluting with 5:1 hexanes/ethyl acetate provides the title compound.

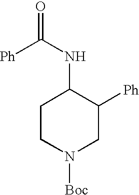

Preparation 2. 4-Benzoylamino-3-phenyl-piperidine-1-carboxylic acid tert-butyl ester. Prepared according to the General Procedures.

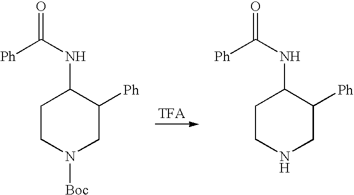

Preparation 3. N-(3-Phenyl-piperidin-4-yl)-benzamide. A solution of 4-benzoylamino-3-phenyl-piperidine-1-carboxylic acid tert-butyl ester (660 mg, 1.74 mmol) in methylene chloride (17 mL) was treated with TFA (3 mL). The mixture was stirred until complete by TLC analysis, at which point the solvent was removed under vacuum. The residue was partitioned between methylene chloride and saturated sodium bicarbonate. The organic layer was isolated, dried and concentrated to give 440 mg (91%) of the title compound as a pale yellow oil.

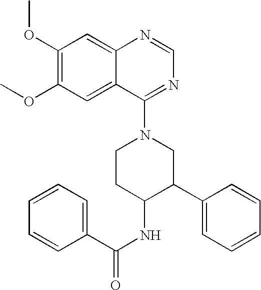

### Example 1

### N-[1-(6,7-Dimethoxy-quinazolin-4-yl)-3-phenyl-piperidin-4-yl]-benzamide

To 4-chloro-6,7-dimethoxyquinazoline (353 mg, 1.57 mmol), prepared as described in Wright, S. W., et al., Anilinoquinazoline inhibitors of fructose 1,6-biphosphatase bind at a novel allosteric site: synthesis, in vitro characterization, and x-ray crystallography. J. Med. Chem., 2002. 45: p. 3865-3877 and N-(3-phenyl-piperidin-4-yl)-benzamide (440 mg, 1.57 mmol) in a mixture of toluene (10 mL) and isopropanol (10 mL) was added potassium carbonate (217 mg, 1.57 mmol). The mixture was heated at reflux until complete by TLC analysis, and then was concentrated under vacuum. The residue was suspended in water and extracted with methylene chloride. The extracts were dried, concentrated, and chromatographed on a silica gel column eluting with ethyl acetate. The product fractions were pooled and concentrated, and the residue was crystallized from ethyl acetate to provide 195 mg (27%) of the title compound as a white powder. Mass spectrum m/e=469.2.

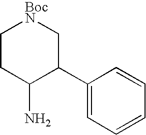

Preparation 4 4-Amino-3-phenyl-piperidine-1-carboxylic acid tert-butyl ester. Prepared according to the General Procedures.

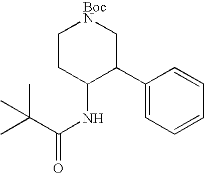

Preparation 5 4-(2,2-Dimethyl-propionylamino)-3-phenyl-piperidine-1-carboxylic acid tert-butyl ester. To 4-amino-3-phenyl-piperidine-1-carboxylic acid tert-butyl ester (333 mg, 1.21 mmol) and potassium carbonate (165 mg, 1.21 mmol) in methylene chloride (12 mL) was added trimethylacetyl chloride (145 mg, 1.21 mmol) followed by 4-N,N-dimethylamino pyridine (0.10 mmol). The mixture was stirred at rt for 20 h, and then was washed with water, dried through cotton, and concentrated. The residue was purified by silica gel chromatography eluting with ethyl acetate to yield 413 mg (95%) of the title compound as a pale yellow foam.

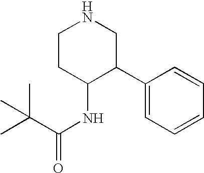

Preparation 6 2,2-Dimethyl-N-(3-phenyl-piperidin-4-yl)-propionamide. This compound was prepared similarly to Preparation 3.

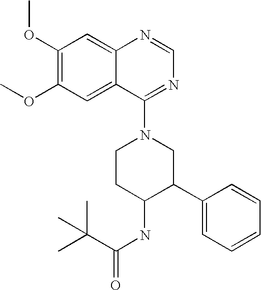

### Example 2

### N-[1-(6,7-Dimethoxy-quinazolin-4-yl)-3-phenyl-piperidin-4-yl]-2,2-dimethyl-propionamide

Prepared similarly to Example 1. Mass spectrum m/e=449.3.

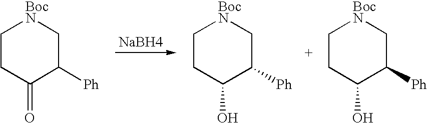

Preparation 7 cis- and trans-N-boc-3-phenyl-4-hydroxypiperidine. To N-Boc-3-phenyl-4-oxopiperidine (3.50 g, 12.7 mmol) in methanol (50 mL) at ice-bath temperature was added sodium borohydride (580 mg, 12.7 mmol). The mixture was stirred for 1 h, and concentrated under vacuum. The mixture was dissolved in methylene chloride, washed with water, dried via filtration through cotton and concentrated. Purification by silica gel chromatography eluting with 3:1 hexanes/ethyl acetate gave the title material.

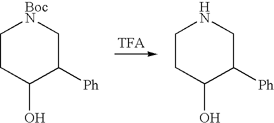

Preparation 8-4-Hydroxy-3-phenylpiperidine. Prepared similarly to Preparation 3.

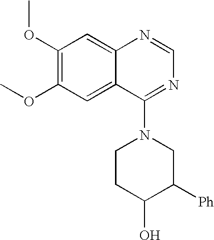

### Example 3

### 1-(6,7-Dimethoxy-quinazolin-4-yl)-3-phenyl-piperidin-4-ol

Prepared similarly to Example 1. Mass spectrum m/e=366.1.

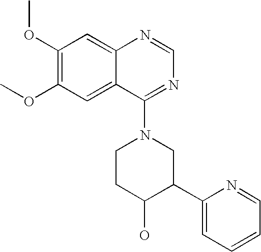

### Example 4

### 1′-(6,7-Dimethoxy-quinazolin-4-yl)-1′,2′,3′,4′,5′,6′-hexahydro-[2,3′]bipyridinyl-4′-ol

Prepared similarly to Example 1. Mass spectrum m/e=367.2.

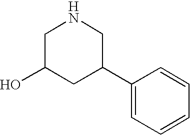

Preparation 10 5-Phenyl-piperidin-3-ol. Prepared according to reported procedures in Great Britain Patent Application GB2060617A.

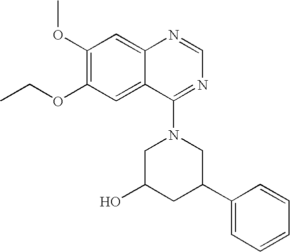

### Example 6

### 1-(6-Ethoxy-7-methoxy-quinazolin-4-yl)-5-Phenyl-piperidin-3-ol

Prepared similarly to Example 1 and using the general procedures for quinazoline synthesis. Mass spectrum m/e=380.1.

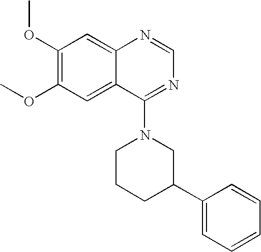

### Example 7

### 1-(6,7-Dimethoxy-quinazolin-4-yl)-5-phenyl-piperidine

Prepared similarly to Example 1. Mass spectrum m/e=350.1.

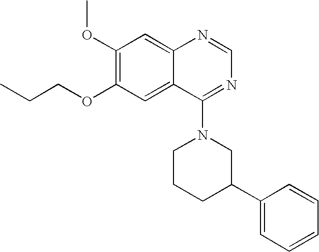

### Example 8

### 7-Methoxy-4-(3-phenyl-Piperidin-1-yl)-6-propoxy-quinazoline

Prepared similarly to Example 1 and using the general procedures for quinazoline synthesis. Mass spectrum m/e=394.1.

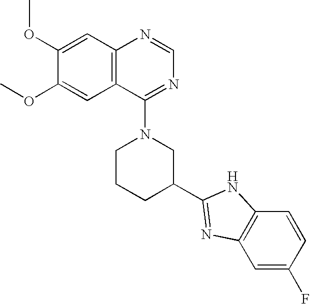

### Example 9

### 4-[3-(5-Fluoro-1H-benzoimidazol-2-yl)-Piperidin-1-yl]-6,7-dimethoxy-quinazoline

Prepared similarly to Example 1.

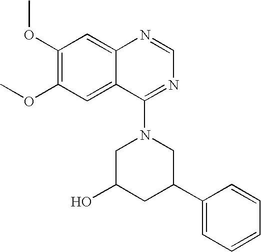

### Example 10

### 1-(6,7-Dimethoxy-quinazolin-4-yl)-5-phenyl-piperidin-3-ol

Prepared similarly to Example 1. Mass spectrum m/e=366.1.

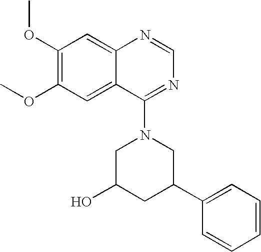

### Example 11

### trans-1-(6,7-Dimethoxy-quinazolin-4-yl)-5-phenyl-piperidin-3-ol

Prepared similarly to Example 1. Mass spectrum m/e=366.1

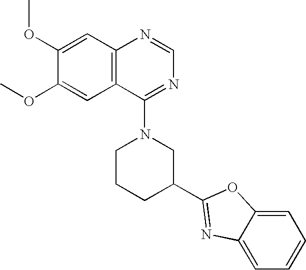

### Example 12

### 4-(3-Benzooxazol-2-yl-piperidin-1-yl)-6,7-dimethoxy-quinazoline

Prepared similarly to Example 1.

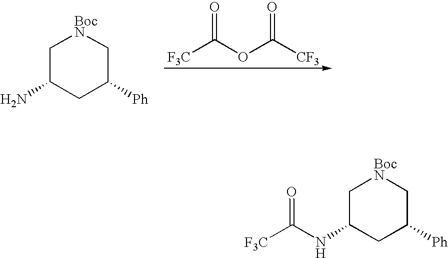

Preparation 11 3-Phenyl-5-(2,2,2-trifluoro-acetylamino)-Piperidine-1-carboxylic acid tert-butyl ester. N-Boc-3-amino-5-phenyl piperidine (879 mg, 3.18 mmol), triethylamine (483 mg, 4.78 mmol) and trifluoroacetic anhydride (670 mg, 3.18 mmol) were stirred in methylene chloride (20 mL) at 0° C. for 30 min, and then at rt for 30 min. The solution was washed with water, dried through cotton, and concentrated. Silica gel chromatography eluting with 9:1 hexanes/ethyl acetate provided 775 mg (66%) of the title compound as a white solid.

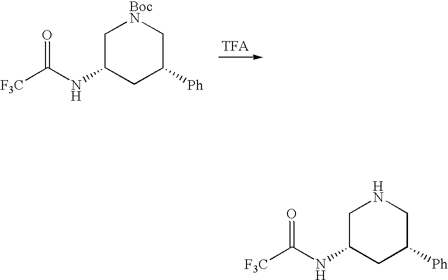

Preparation 12 2,2,2-Trifluoro-N-(5-phenyl-piperidin-3-yl)-acetamide. Prepared similarly to Preparation 3.

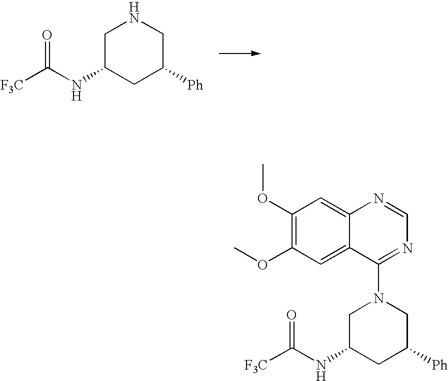

Preparation 13 N-[1-(6,7-Dimethoxy-quinazolin-4-yl)-5-phenyl-piperidin-3-yl]-2,2,2-trifluoro-acetamide. Prepared similarly to Example 1.

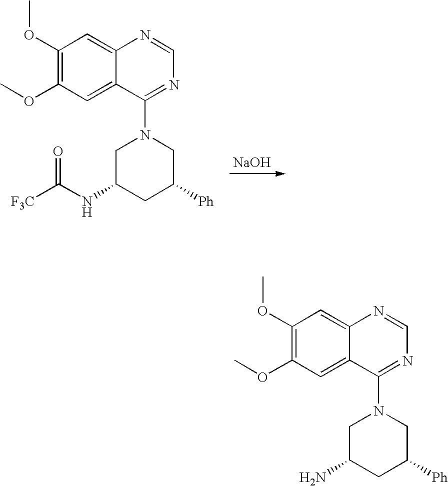

### Example 13

### 1-(6,7-Dimethoxy-quinazolin-4-yl)-5-phenyl-Piperidin-3-ylamine hydrochloride

A sample of N-[1-(6,7-dimethoxy-quinazolin-4-yl)-5-phenyl-piperidin-3-yl]-2,2,2-trifluoro-acetamide (287 mg, 0.62 mmol) was stirred in methanol (6 mL) and 3 M NaOH (6 mL) at rt for 2 h. The methanol was removed under vacuum and the aqueous remainder was extracted 3× with methylene chloride. The combined extracts were dried through cotton and concentrated to yield 212 mg of a white foam. The foam was then dissolved in isopropanol, and 1.0 equivalent of concentrated HCl was added with stirring. The mixture was then concentrated in vacuo to give the title compound as a white powder. Mass spectrum m/e=365.2.

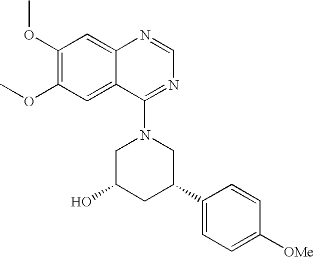

### Example 14

### 1-(6,7-Dimethoxy-quinazolin-4-yl)-5-(4-methoxy-phenyl)-piperidin-3-ol

Prepared similarly to Example 1. Mass spectrum m/e=396.2.

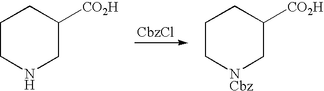

Preparation 14 Piperidine-1,3-dicarboxylic acid 1-benzyl ester. To a stirred solution of 3-piperidine carboxylic acid (1.48 g, 11.5 mmol) and saturated sodium bicarbonate (40 mL) in tetrahydrofuran (40 mL) at 0° C. was added benzylchloroformate (2.05 g, 12.0 mmol). The mixture was stirred in an ice-bath for 3 h, and then at room temperature for 16 h. The mixture was then cooled to 0° C. and the pH was reduced to ca. 1.0 with 6 M HCl. The mixture was extracted three times with ethyl acetate. The combined extracts were dried with magnesium sulfate, filtered, and concentrated to provide 10.0 g of the title compound as a colorless oil.

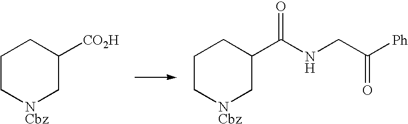

Preparation 15 3-(2-Oxo-2-phenyl-ethylcarbamoyl)-piperidine-1-carboxylic acid benzyl ester. A mixture of piperidine-1,3-dicarboxylic acid 1-benzyl ester (3.0 g, 11.4 mmol), triethylamine (4.62 g, 45.6 mmol), and 1-propanphosphonic acid anhydride (3.63 g, 11.4 mol, 6.80 mL of a 50% w/w solution in ethyl acetate) and 2 aminoacetophenone hydrochloride (1.96 g, 11.4 mmol) in THF (55 mL) was stirred at rt for 16 h. The mixture was then concentrated, and the residue was dissolved in CH₂Cl₂. The solution was washed with 1 M NaOH, dried through cotton, and concentrated. Silica gel chromatography eluting with 1:2 hexanes/ethyl acetate gave the title compound as a pale yellow solid.

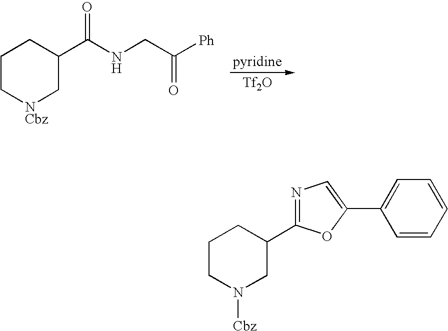

Preparation 16 3-(5-Phenyl-oxazol-2-yl)-piperidine-1-carboxylic acid benzyl ester. To 3-(2-oxo-2-phenyl-ethylcarbamoyl)-piperidine-1-carboxylic acid benzyl ester (2.71 g, 7.13 mmol) and pyridine (1.13 g, 14.3 mmol), in methylene chloride (70 mL) at rt was added dropwise trifluoromethane sulfonic anhydride (2.21 g, 282 mmol). (exothermic reaction.) The solution was stirred for 3 h, and was then washed with 1 M HCl, filtered through cotton, and concentrated. Silica gel chromatography eluting with 1:1 hexanes/ethyl acetate provided 2.40 g (93%) of the title compound as a clear brown oil.

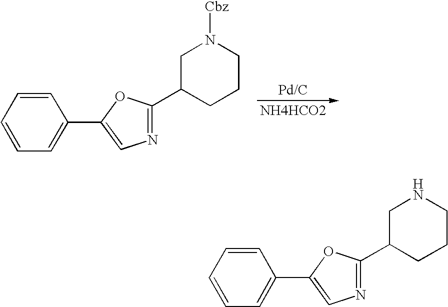

Preparation 17 3-(5-Phenyl-oxazol-2-yl)-Piperidine. A mixture of 3-(5-phenyl-oxazol-2-yl)-piperidine-1-carboxylic acid benzyl ester (2.40 g, 6.63 mmol), 10% palladium on carbon (100 mg), and ammonium formate (4.18 g, 66.3 mmol) was heated in ethanol (33 mL) at 60° C. for 20 h. The mixture was filtered through Celite and concentrated. The residue was dissolved in methylene chloride and the resultant solution was washed with water, dried through cotton, and concentrated to give 1.41 g (94%) of a yellow oil. The oil was dissolved in hot ethyl acetate and 1.0 equivalent of p-toluenesulfonic acid monohydrate was added. After stirring for 24 h the solids were collected via filtration and dried under vacuum to give 1.91 g (72%) of the title compound as a white powder.

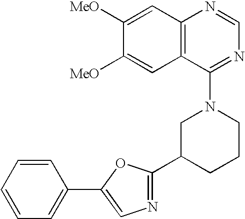

### Example 15

### 6,7-Dimethoxy-4-[3-(5-phenyl-oxazol-2-yl)-piperidin-1-yl]-quinazoline

Prepared similarly to Example 1. Mass spectrum m/e calc. for M+H=417.2.

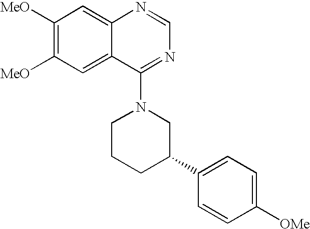

### Example 16

### 6,7-Dimethoxy-4-[3-(4-methoxy-Phenyl)-piperidin-1-yl]-quinazoline

Prepared similarly to Example 1. Mass spectrum m/e=380.2

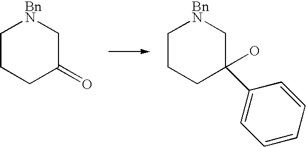

Preparation 18 1-Benzyl-3-phenyl-piperidin-3-ol. 1-Benzyl-3-piperidine hydrochloride hydrate (1.02 g, 5.40 mmol) was suspended in methylene chloride, washed with 1 M NaOH, dried through cotton, and concentrated to give 1.02 g of free base material. The free base was dissolved in THF (40 mL) and cooled to 0° C. Phenyl magnesium bromide (3.0 M in ether, 8.10 mmol, 2.70 mL) was added dropwise over 30 min, at which point the solution was warmed to rt and stirred for 3 h. The mixture was then concentrated and the residue dissolved in methylene chloride. The resultant solution was washed with 10% saturated NH4Cl, dried through cotton and concentrated. Silica gel chromatography eluting with hexanes/ethyl acetate (3:1) gave 0.975 g of the title compound as a pale yellow oil.

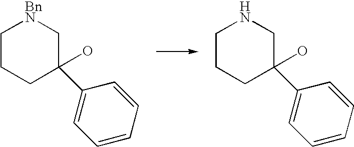

Preparation 19 3-Phenyl-piperidin-3-ol. A mixture of 1-benzyl-3-phenyl-piperidin-3-ol (975 mg, 3.65 mmol), 10% palladium on carbon (250 mg) and 12 M HCl (4.02 mmol, 0.335 mL) in ethanol (50 mL) was hydrogenated at 45 psi on a Par shaker for 4 h. The mixture was carefully filtered through Celite and concentrated to give an off-white solid. The material was crystallized from isopropanol to provide 375 mg (48%) of the title compound as a white solid.

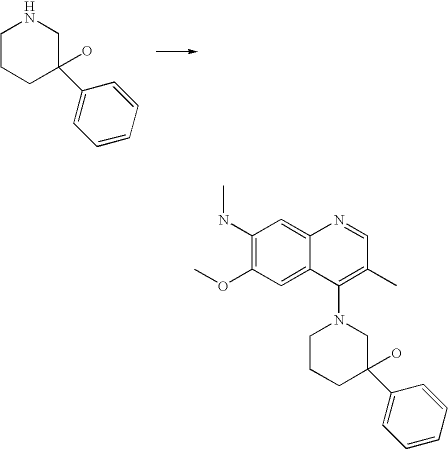

### Example 17

### 1-(6,7-Dimethoxy-quinazolin-4-yl)-3-phenyl-piperidin-3-ol

Prepared similarly to Example 1. Mass spectrum m/e=366.2.

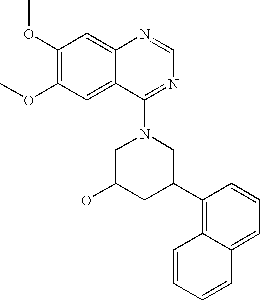

### Example 18

### -1-(6,7-Dimethoxy-quinazolin-4-yl)-5-naphthalen-1-yl-piperidin-3-ol

Prepared similarly to Example 1. Mass spectrum m/e calc. for M+H=416.2.

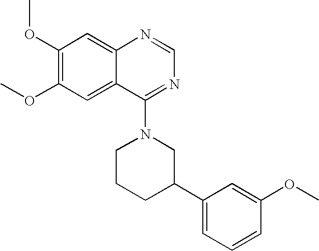

### Example 19

### 6,7-Dimethoxy-4-[3-(3-methoxy-phenyl)-piperidin-1-yl]-quinazoline. Prepared similarly to Example 1. Mass spectrum m/e=380.1

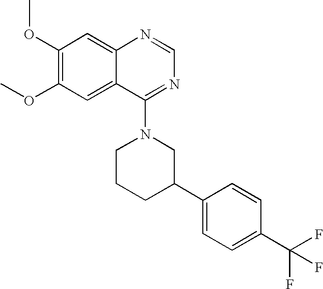

### Example 20

### 6,7-Dimethoxy-4-[3-(4-trifluoromethyl-Phenyl)-piperidin-1-yl]-quinazoline

Prepared similarly to Example 1. Mass spectrum m/e calc. for M+H=418.2

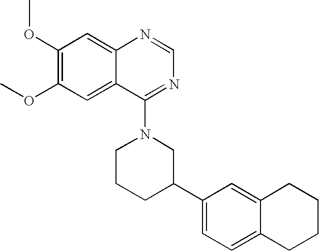

### Example 21

### 6,7-Dimethoxy-4-[3-(5,6,7,8-tetrahydro-naphthalen-2-yl)-piperidin-1-yl]-quinazoline

Prepared similarly to Example 1. Mass spectrum m/e=404.3.

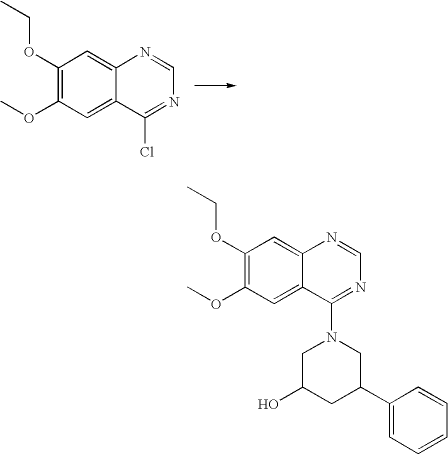

Preparation 20 1-(7-Ethoxy-6-methoxy-quinazolin-4-yl)-5-phenyl-piperidin-3-ol hydrochloride. To 4-chloro-7-ethoxy-6-methoxy-quinazoline (120 mg, 0.5 mmol) in 3 mL of toluene and 3 mL of isopropanol was added potassium carbonate (138 mg, 1 mmol) and 5-phenyl-piperidin-3-o (106 mg). The mixture was refluxed for 25 h, and then diluted with water and extracted 3× with ethyl acetate. The combined extracts were washed with brine and concentrated. Silica gel chromatography eluting with 2:98 ethanol/ethyl acetate provided the free base of the title compound. Treatment with a 1 M solution of HCl in ether provided the title compound in the amount of 51 mg (27%).

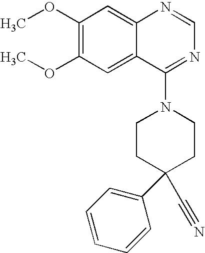

### Example 22

### 1-(6,7-Dimethoxy-quinazolin-4-yl)-4-phenyl-piperidine-4-carbonitrile

Prepared similarly to Example 1 using commercially available 4-cyano-4-phenylpiperidine.

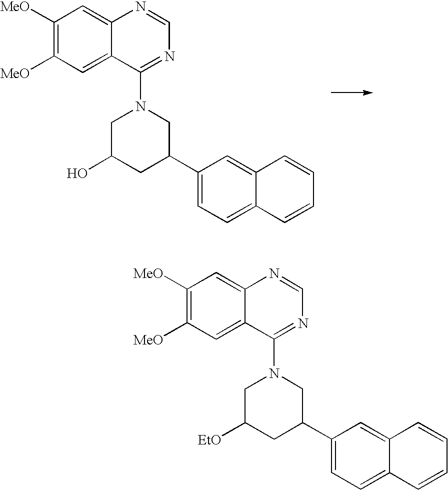

### Example 23

### 4-(3-Ethoxy-5-naphthalen-2-yl-piperidin-1-yl)-6,7-dimethoxy-quinazoline

To a mixture of 1-(6,7-dimethoxy-quinazolin-4-yl)-5-naphthalen-2-yl-piperidin-3-ol (65 mg, 0.126 mmol) in dimethylformamide (3 mL) was added sodium hydride (18 mg, 0.75 mmol). The mixture was stirred for 10 min and diethyl sulfate (25 mg, 0.164 mmol) was added. The mixture was heated to 600C for 2 h, and was then quenched with water. After stirring at 60° C. for 15 min, the solution was extracted twice with ethyl acetate and the combined extracts were washed with brine. To the extracts was added a slight excess of 4 M HCl, and the mixture was concentrated. The solid residue was crystallized from ethyl acetate/ether to provide 43 mg (72%) of the title compound as a white powder. MS 444.4.

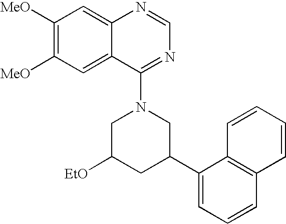

### Example 24

### 4-(3-Ethoxy-5-naphthalen-1-yl-piperidin-1-yl)-6,7-dimethoxy-quinazoline

prepared similarly. MS 444.4.

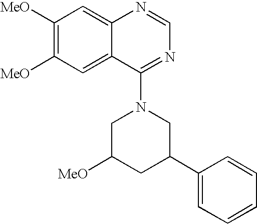

### Example 25

### 6,7-Dimethoxy-4-(3-methoxy-5-phenyl-piperidin-1-yl)-quinazoline

Prepared similarly. MS 380.3.

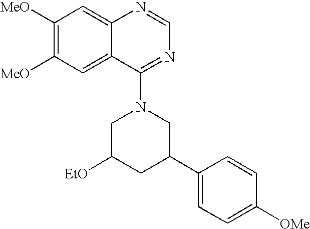

### Example 26

### 4-[3-Ethoxy-5-(4-methoxy-phenyl)-piperidin-1-yl]-6,7-dimethoxy-quinazoline

Prepared similarly. MS 424.4.

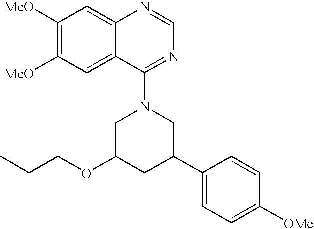

### Example 27

### 6,7-Dimethoxy-4-[3-(4-methoxy-phenyl)-5-propoxy-piperidin-1-yl]-quinazoline

Prepared similarly. MS 438.4.

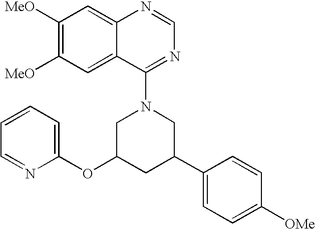

### Example 28

### 6,7-Dimethoxy-4-[3-(4-methoxy-phenyl)-5-(pyridin-2-yloxy)-piperidin-1-yl]-quinazoline

Prepared similarly. MS 473.3.

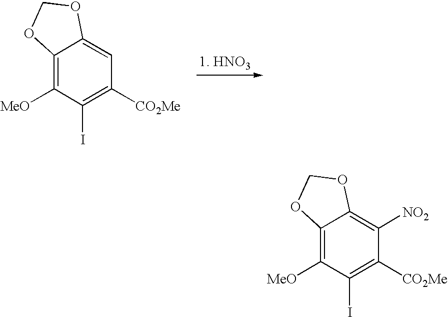

Preparation 21. Methyl 6-iodo-7-methoxy-4-n itrobenzo[d][1,3]dioxole-5-carboxylate. To a solution of nitric acid (30 ml) solvent was added 6-iodo-7-methoxy-benzo[1,3]dioxole-5-carboxylic acid methyl ester (1.4 g, 4.2 mol) at 0° C. The reaction mixture was stirred for 1 h and then poured into crushed ice. The resultant solid was collected via filtration and dried under vacuum to provide 1.3 g (82%) of the title compound. ¹H NMR δ: 3.87 (s, 3H), 4.05 (s, 3H), 6.16 (s, 2H).

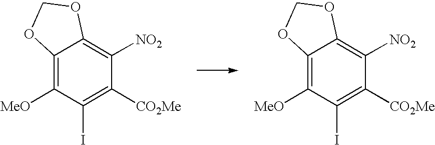

Preparation 22. Methyl 4-amino-7-methoxybenzo[d][1,3]dioxole-5-carboxylate. 6-iodo-7-methoxy-benzo[1,3]dioxole-5-carboxylic acid methyl ester (0.78 g, 2 mmol) was hydrogenated over 20% palladium hydroxide on carbon in the presence of excess ammonium formate in MeOH (15 mL) for 2 h. Upon completion the reaction mixture was filtrated through Celite and concentrated. The resultant solid was extracted with methylene chloride and the extract was concentrated to give a pale-yellow solid. Recrystallization from MeOH provided 370 mg, (82%) of the title compound. ¹H NMR 6: 3.80 (s, 3H), 3.97 (s, 3H), 5.87 (s, 2H), 6.99 (s, 1H, Ar—H).

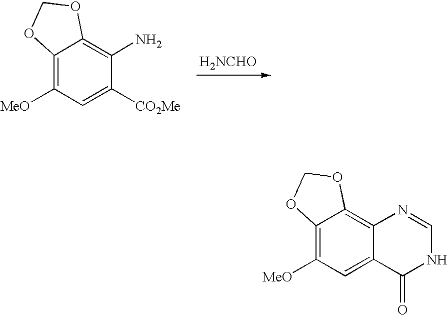

Preparation 23. 4-Methoxy-[1,3]dioxolo[4,5-h]quinazolin-6 (7H)-one. A mixture of methyl 4-amino-7-methoxybenzo[d][1,3]dioxole-5-carboxylate (0.35 g, 1.55 mmol) and ammonium carbonate (0.24 g, 3.1 mmol) in formamide (3 ml) was stirred at 170° C. and for 24 h. The reaction mixture was poured into crushed ice and stored overnight. The resultant solid was collected and dried to give 150 mg (44%) of a brown solid. ¹H NMR δ: 4.12 (s, 3H), 6.16 (s, 2H), 7.21 (s, 1H), 7.95 (s, 1H).

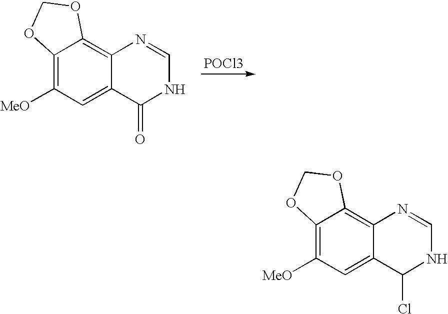

Preparation 24. 6-Chloro-4-methoxy-[1,3]dioxolo[4,5-h]quinazoline. 4-Methoxy-[1,3]dioxolo[4,5-h]quinazolin-6 (7H)-one was refluxed in a solution of POCl₃ and SOCl₂ (5:2) for 3 h. After the removal of solvent, phosphate buffer (pH=7.0) was added. The resultant solution was extracted 3× with CH₂Cl₂. The CH₂Cl₂ extracts were then dried and concentrated to give the title compound as a pale-yellow solid.

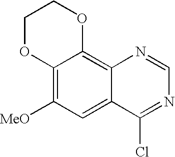

Preparation 25. 8-Chloro-10-methoxy-2,3-dihydro-1,4-dioxa-5,7-diaza-phenanthrene. Prepared similarly, according to Scheme 3.

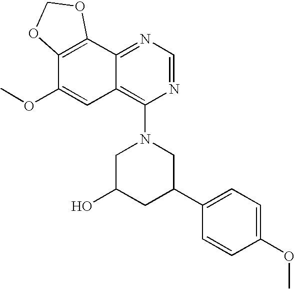

### Example 29

### 1-(4-Methoxy-1,3-dioxa-7,9-diaza-cyclopenta[a]naphthalen-6-yl)-5-(4-methoxy-phenyl)-piperidin-3-ol

Prepared similarly to Example 1 substituting 6-chloro-4-methoxy-[1,3]dioxolo[4,5-h]quinazoline for 4-chloro-6,7-dimethoxyquinazoline.

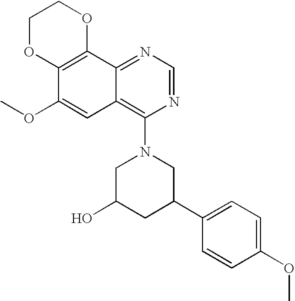

### Example 30

### 1-(10-Methoxy-2,3-dihydro-1,4-dioxa-5,7-diaza-Phenanthren-8-yl)-5-(4-methoxy-phenyl)-piperidin-3-ol

Prepared similarly to Example 1 substituting 8-chloro-10-methoxy-2,3-dihydro-1,4-dioxa-5,7-diaza-phenanthrene for 4-chloro-6,7-dimethoxyquinazoline.

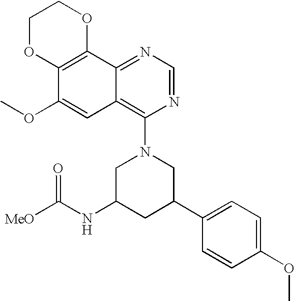

### Example 31

### [1-(10-Methoxy-2,3-dihydro-1,4-dioxa-5,7-diaza-phenanthren-8-yl)-5-(4-methoxy-phenyl)-piperidin-3-yl]-carbamic acid methyl ester

Prepared similarly to Example 30.

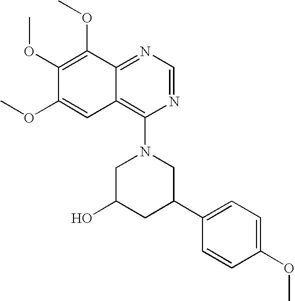

### Example 32

### 5-(4-Methoxy-phenyl)-1-(6,7,8-trimethoxy-quinazolin-4-yl)-piperidin-3-ol

Prepared similarly to Example 1 substituting 4-chloro-6,7,8-trimethoxyquinazoline, which was prepared similarly to the procedure in Takase, Y., et al., Cyclic GMP Phosphodiesterase inhibitors, The discovery of a novel potent inhibitor, 4-((3,4-(methylenedioxy)benzyl)amino)-6,7,8-trimethoxyquinazoline. J. Med. Chem., 1993. 36(36): p. 3675-3770, for 4-chloro-6,7-dimethoxyquinazoline.

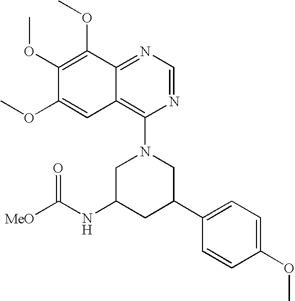

### Example 33

### [5-(4-Methoxy-phenyl)-1-(6,7,8-trimethoxy-quinazolin-4-yl)-piperidin-3-yl]-carbamic acid methyl ester

Prepared similarly to Example 32.

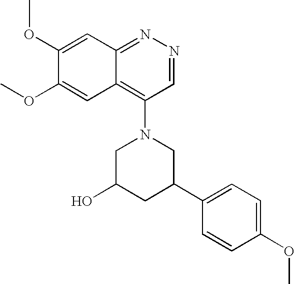

### Example 34

### 1-(6,7-Dimethoxy-cinnolin-4-yl)-5-(4-methoxy-phenyl)-Piperidin-3-ol

Prepared similarly to Example 1 substituting 4-chloro-6,7-dimethoxycinnoline, which was prepared similar to the procedure in Castle, R. N. and F. H. Kruse, Cinnoline Chemistry. I. Some condensation reactions of 4-chlorocinnoline. J. Org. Chem., 1952. 17: p. 1571-1575, for 4-chloro-6,7-dimethoxyquinazoline.

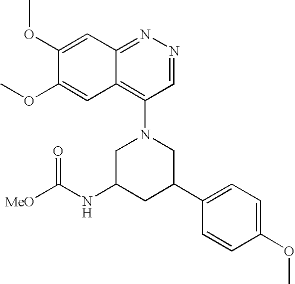

### Example 35

### [1-(6,7-Dimethoxy-cinnolin-4-yl)-5-(4-methoxy-phenyl)-piperidin-3-yl]-carbamic acid methyl ester

Prepared similarly to Example 34.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed:

1. A compound having the formula 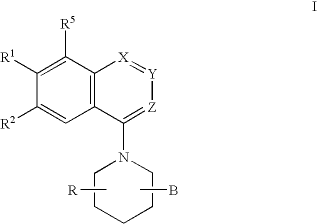
or a pharmaceutically acceptable salt, solvate or prodrug thereof,
wherein X, Y and Z are each independently N or CH, provided that at least one of X, Y and Z must be N or CH and provided that when Z is nitrogen, Y is CH; and when Y is nitrogen, X is nitrogen and Z is CH;
wherein R¹, R² and R⁵ are independently H, halogen, —CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹, R² and R⁵ are independently alkoxy, alkenyloxy or alkyl, R¹ and R² or R¹ and R⁵ may optionally be connected to form a 5 to 8 membered ring; and when R¹, R² and R⁵ are —NR³R⁴, R³ and R⁴ may optionally combine with the nitrogen in which they are attached to form a 5 to 8 membered ring;
wherein R is H, —COOR³, —CONR³R⁴, —COR⁴, —NR³R⁴, —NHCOR³, —OH, —HNCOOR³, —CN, —HNCONHR⁴, (C₁-C₆)alkyl or (C₂-C₆) alkoxy;
wherein R³ and R⁴ are independently H, (C₁-C₆) alkyl, alkenyl, aryl or substituted aryl;
wherein B is hydrogen, phenyl, naphthyl, or a 5- to 6-membered heteroaryl ring, optionally fused to a benzo group or heteroaryl ring, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, (C₁-C₈) alkoxy, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂XOH with an ortho —COOH, wherein t is one, two or three; (b)-CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷, wherein v is zero, one, two or three and —COR¹⁶ and R¹⁷ taken together with the nitrogen to which they are attached may form a 4- to 6-membered lactam ring.

2. The compound of claim 1 wherein B is phenyl, phenyl substituted by (C₁-C₅)alkoxy, (C₁-C₅)alkyl, trifluoroalkyl or (C₂-C₅)trifluoroalkoxy.

3. The compound of claim 2 wherein B is phenyl substituted with trifluoromethyl.

4. The compound of claim 2 wherein R is hydrogen, (C₁-C₅)alkoxy, —NR³R⁴, —HNCOOR³, or hydroxyl.

5. The compound of claim 2 wherein R¹ and R² are each independently (C₁-C₆)alkoxy.

6. The compound of claim 5 wherein R¹ and R² are each ethoxy or methoxy.

7. The compound of claim 1 wherein R¹ and R² are each independently (C₁-C₆)alkoxy, X and Z are N, Y is CH, B is phenyl or substituted phenyl and R is —NHCOR³.

8. The compound of claim 7 wherein R¹ is methoxy when R² is ethoxy or R¹ is ethoxy when R² is methoxy.

9. The compound of claim 1 wherein said heteroaryl group in substituent B is a heteroaryl or benzo-fused heteroaryl group selected from pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

10. A compound according to claim 1 selected from the group consisting of:
N-[1-(6,7-Dimethoxy-quinazolin-4-yl)-3-phenyl-piperidin-4-yl]-benzamide;
N-[1-(6,7-Dimethoxy-quinazolin-4-yl)-3-phenyl-piperidin-4-yl]-2,2-dimethyl-propionamide;
cis-1-(6,7-Dimethoxy-quinazolin-4-yl)-3-phenyl-piperidin-4-ol;
trans-1-(6,7-Dimethoxy-quinazolin-4-yl)-3-phenyl-piperidin-4-ol;
1′-(6,7-Dimethoxy-quinazolin-4-yl)-1′,2′,3′,4′,5′,6′-hexahydro-[2,3′]bipyridinyl-4′-ol;
1-(6-Ethoxy-7-methoxy-quinazolin-4-yl)-5-phenyl-piperidin-3-ol;
1-(6,7-Dimethoxy-quinazolin-4-yl)-5-phenyl-piperidine;
7-Methoxy-4-(3-phenyl-piperidin-1-yl)-6-propoxy-quinazoline;
4-[3-(5-Fluoro-1H-benzoimidazol-2-yl)-piperidin-1-yl]-6,7-dimethoxy-quinazoline;
1-(6,7-Dimethoxy-quinazolin-4-yl)-5-phenyl-piperidin-3-ol;
trans-1-(6,7-Dimethoxy-quinazolin-4-yl)-5-phenyl-piperidin-3-ol;
4-(3-Benzooxazol-2-yl-piperidin-1-yl)-6,7-dimethoxy-quinazoline;
1-(6,7-Dimethoxy-quinazolin-4-yl)-5-phenyl-piperidin-3-ylamine hydrochloride;
1-(6,7-Dimethoxy-quinazolin-4-yl)-5-(4-methoxy-phenyl)-piperidin-3-ol;
6,7-Dimethoxy-4-[3-(5-phenyl-oxazol-2-yl)-piperidin-1-yl]-quinazoline;
6,7-Dimethoxy-4-[3-(4-methoxy-phenyl)-piperidin-1-yl]-quinazoline;
1-(6,7-Dimethoxy-quinazolin-4-yl)-3-phenyl-piperidin-3-ol;
cis-1-(6,7-Dimethoxy-quinazolin-4-yl)-5-naphthalen-1-yl-piperidin-3-ol;
6,7-Dimethoxy-4-[3-(3-methoxy-phenyl)-piperidin-1-yl]-quinazoline;
6,7-Dimethoxy-4-[3-(4-trifluoromethyl-phenyl)-piperidin-1-yl]-quinazoline;
6,7-Dimethoxy-4-[3-(5,6,7,8-tetrahydro-naphthalen-2-yl)-piperidin-1-yl]-quinazoline;
1-(6,7-Dimethoxy-quinazolin-4-yl)-4-phenyl-piperidine-4-carbonitrile;
1-(4-Methoxy-1,3-dioxa-7,9-d iaza-cyclopenta[a]naphthalen-6-yl)-5-(4-methoxy-phenyl)-piperidin-3-ol;
1-(10-Methoxy-2,3-dihydro-1,4-dioxa-5,7-diaza-phenanthren-8-yl)-5-(4-methoxy-phenyl)-piperidin-3-ol;
[1-(10-Methoxy-2,3-dihydro-1,4-dioxa-5,7-diaza-phenanthren-8-yl)-5-(4-methoxy-phenyl)-piperidin-3-yl]-carbamic acid methyl ester;
5-(4-Methoxy-phenyl)-1-(6,7,8-trimethoxy-quinazolin-4-yl)-piperidin-3-ol;
[5-(4-Methoxy-phenyl)-1-(6,7,8-trimethoxy-quinazolin-4-yl)-piperidin-3-yl]-carbamic acid methyl ester;
1-(6,7-Dimethoxy-cinnolin-4-yl)-5-(4-methoxy-phenyl)-piperidin-3-ol;
[1-(6,7-Dimethoxy-cinnolin-4-yl)-5-(4-methoxy-phenyl)-piperidin-3-y]-carbamic acid methyl ester;
and pharmaceutical acceptable salts thereof.

11. A pharmaceutical composition for treating psychotic disorders, delusional disorders and drug induced psychosis; anxiety disorders, movement disorders, mood disorders, neurodegenerative disorders and drug addiction, comprising an amount of a compound of formula I according to claim 1 effective in treating said disorder or condition.

12. A method of treating a disorder selected from psychotic disorders, delusional disorders and drug induced psychosis; anxiety disorders, movement disorders, mood disorders, and neurodegenerative disorders, which method comprises administering an amount of a compound of claim 1 effective in treating said disorder.

13. The method of claim 12, wherein said disorder are selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.

14. A compound having the formula 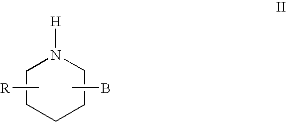
wherein R is H, —COOR³, —CONR³R⁴, —COR⁴, —NR³R⁴, —OH, —HNCOOR³, —CN, —HNCONHR⁴, (C₁-C₆)alkyl, (C₂-C₆) alkoxy, or (C₂-C₆)trifluoroalkoxy;
wherein R³ and R⁴ are independently H, (C₁-C₆) alkyl, (C₂-C₈)alkenyl, aryl or substituted aryl.
wherein B is hydrogen, phenyl, naphthyl or a 5- to 6-membered heteroaryl ring, optionally fused to a benzo group, containing from one to four heteroatoms in the ring selected from oxygen, nitrogen and sulfur, with the proviso that said ring cannot contain two adjacent oxygen atoms or two adjacent sulfur atoms and wherein each of the foregoing phenyl, naphthyl and heteroaryl rings may optionally be substituted with one to three substituents independently selected from (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups;
wherein B is a phenyl, naphthyl, heteroaryl or benzo-fused heteroaryl ring, each said ring may be optionally substituted with one to three substituents independently selected from phenyl, naphthyl and a 5- to 6-membered heteroaryl ring containing from one to four hetero-atoms selected from oxygen, nitrogen and sulfur, with the proviso that said heteroaryl ring cannot contain two adjacent oxygen atoms or two adjacent sulfur atoms, and wherein each independently selected phenyl, naphthyl or heteroaryl substituent may itself be substituted with from one to three (C₁-C₈)alkyl or C₃-C₈ cycloalkyl substituents, wherein examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl;
when B is a phenyl, naphthyl or heteroaryl ring, each said ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷ wherein v is zero, one, two or three and —COR¹⁶ and R¹⁷ taken together with the nitrogen to which they are attached may form a 4- to 6-membered lactam ring.

15. A process for preparing a compound of the formula 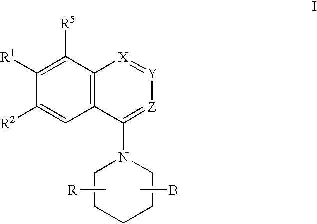
or a pharmaceutically acceptable salt, solvate or prodrug thereof,
wherein X, Y and Z are each independently N or CH, provided that at least one of X, Y and Z must be N or CH and provided that when Z is nitrogen, Y is CH; and when Y is nitrogen, X is nitrogen and Z is CH;
wherein R¹, R² and R⁵ are independently H, halogen, —CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹, R² and R⁵ are independently alkoxy, alkenyloxy or alkyl, R¹ and R² or R¹ and R⁵ may optionally be connected to form a 5 to 8 membered ring; and when R¹, R² and R⁵ are —NR³R⁴, R³ and R⁴ may optionally combine with the nitrogen in which they are attached to form a 5 to 8 membered ring;
wherein R is H, —COOR³, —CONR³R⁴, —COR⁴, —NR³R⁴, —NHCOR³, —OH, —HNCOOR³, —CN, —HNCONHR⁴, (C₁-C₆)alkyl or (C₂-C₆) alkoxy;
wherein R³ and R⁴ are independently H, (C₁-C₆) alkyl, alkenyl, aryl or substituted aryl;
wherein B is hydrogen, phenyl, naphthyl, or a 5- to 6-membered heteroaryl ring, optionally fused to a benzo group, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, (C₁-C₈) alkoxy, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷ wherein v is zero, one, two or three and —COR¹⁶ and R¹⁷ taken together with the nitrogen to which they are attached may form a 4- to 6-membered lactam ring.
comprising reacting a compound of formula IIa 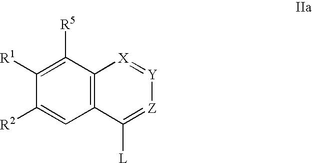
wherein L is a suitable leaving group;
with a compound of formula II 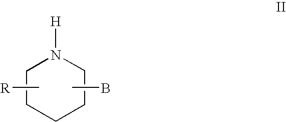
wherein R¹, R², R⁵, X, Y, Z, R and B are defined above.

16. The process of claim 15 wherein L is a leaving group comprising a halogen atom selected from chlorine, bromine and iodine.

17. A compound having the formula 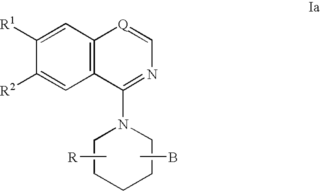
or a pharmaceutically acceptable salt, solvate or prodrug thereof,
wherein Q is N or CH;
wherein R¹ and R² are independently H, halogen, —CN, —COOH, —COOR³, —CONR³R⁴, —COR³, —NR³R⁴, —OH, —NO₂, —(C₆-C₁₄)aryl, 5 to 12 membered heteroaryl, (C₁-C₉)alkyl, (C₁-C₉)alkoxy (C₂-C₉) alkenyl, (C₂-C₉) alkenyloxy (C₂-C₉) alkynyl or (C₃-C₉) cycloalkyl; wherein said alkyl, alkenyl, alkenyloxy, alkynyl, and alkoxy are optionally independently substituted with from 1 to 3 halogens; and when R¹ and R² are independently alkoxy, alkenyloxy or alkyl, R¹ and R² may optionally be connected to form a 5 to 8 membered ring; and when R¹ and R² are —NR³R⁴, R³ and R⁴ may optionally combine with the nitrogen in which they are attached to form a 5 to 8 membered ring;
wherein R is H, —COOR³, —CONR³R⁴, —COR⁴, —NR³R⁴, —NHCOR³, —OH, —HNCOOR³, —CN, —HNCONHR⁴ (C₁-C₆)alkyl or —O(C₂-C₆) alkyl;
wherein R³ and R⁴ are independently H, (C₁-C₆) alkyl, aryl or substituted aryl;
wherein B is hydrogen, phenyl, naphthyl, or a 5- to 6-membered heteroaryl ring, optionally fused to a benzo group, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups; or
when B is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷ wherein v is zero, one, two or three and —COR¹⁶ and R¹⁷ taken together with the nitrogen to which they are attached form a 4- to 6-membered lactam ring.
